(19) United States
(12) Patent Application Publication
(10) Pub. No.: US 2023/0337952 A1
(43) Pub. Date: Oct. 26, 2023

(54) SYSTEM AND METHOD FOR TREATING POST TRAUMATIC STRESS DISORDER (PTSD) AND PHOBIAS

(71) Applicant: WAJI, LLC, REDMOND, WA (US)

(72) Inventors: Matthew EMMA, Redmond, WA (US); David BONANNO, Redmond, WA (US); Robert EMMA, Redmond, WA (US); Amber DENNIS, Redmond, WA (US); Lucera COX, Redmond, WA (US)

(21) Appl. No.: 18/001,474

(22) PCT Filed: Jun. 14, 2021
(86) PCT No.: PCT/IB2021/055230

§ 371 Date: Dec. 12, 2022

Related U.S. Application Data
(60) Provisional application No. 63/038,368, filed on Jun. 12, 2020.

(51) Int. Cl. A61B 5/16; G16H 20/70; A61B 5/00

(52) U.S. Cl. CPC A61B 5/163; A61B 5/165; G16H 20/70; A61B 5/4836

(57) ABSTRACT

A system and method including software and any associated hardware components, for use as a medical device to provide a therapeutic treatment where current clinical practices are less accessible and/or less desirable for the user. In one embodiment the therapeutic treatment is a psychological treatment, such as treatment of post-traumatic stress disorder (PTSD) and or phobia(s). In one embodiment, this is a user-directed treatment.

### FIELD OF THE INVENTION

The present invention relates to a system and method for treating mental health conditions and in particular, to such a system and method for treating such conditions through a guided, staged treatment process.

### BACKGROUND OF THE INVENTION

Many people suffer from PTSD and phobias, but not all have access to treatment. Current treatments require highly skilled psychologists and/or psychiatrists (if pharmaceuticals are recommended). These treatments are very effective but also limit access. In addition, sufferers may need regular treatment, which also decreases access. Access may also be limited by the personal desires of sufferers, who may not wish to visit a therapist of any type or of an available type, for example due to concerns over privacy or due to lack of comfort in such a visit, and/or may not wish to take medication.

Attempts have been made to provide software which is suitable for assisting sufferers with PTSD and phobias. Various references discuss these different types of software. However, such software is currently not able to provide a highly effective treatment. For example, the software does not provide an overall treatment process that supports the underlying treatment method. Other software requires the presence of a therapist to actively guide the therapeutic method.

For example, US20200086077A1 describes treatment of PTSD by using EMDR (Eye Movement Desensitization and Reprocessing) therapy. EMDR requires a therapist to interact with a patient through guided therapy. A stimulus is provided to the user (patient), which may be visual, audible or tactile. This stimulus is provided through some type of hardware device, which may be a computer. The therapist controls the provision of the stimulus to the user's computer. The process described is completely manual.

### SUMMARY OF THE INVENTION

The present invention overcomes the drawbacks of the background art by providing, in at least some embodiments, a system and method for treatment of PTSD and phobias, and optionally for treatment of additional psychological disorders. PTSD and phobias are both suitable for such treatment because they are both characterized by learned or conditioned excessive fears, whether such excessive fears are consciously understood by the user or are subconsciously present. Of course, mixed disorders that feature elements of learned or conditioned excessive fears would be expected to be suitable targets for treatment with your current innovative software and system.

The software may be provided as an app on a mobile phone or may be operated through a desktop or laptop computer. The software is designed for user interaction and participation. The system may use commodity hardware, which is typically available on a mobile phone or computer, such as a mouse, keyboard, touch screen and camera. The device comprises a display screen for displaying a light or other on-screen object for the user's eyes to track. The software instructs the user to maintain tracking of the on-screen object while engaging with a guided plurality of stages for the treatment process.

Preferably, the system includes eye-tracking sensors for determining the tracking of the user's eyes on the displayed light or other on-screen object. Such eye-tracking sensors may comprise for example a video camera for tracking the iris, pupil and/or other component of the eye, to determine the direction of the user's eye gaze.

The system may also include wearables for the recording and collection of biometric data, which will enable further user engagement with the system. A non-limiting example of such a wearable is a heart rate and function measurement device, such as a sports watch wearable.

Various software components are preferred in order to ensure user interaction, such as an animated ball or other client-customized stimulus that moves around the screen and that induces eye movements. The user tracks the visual stimulus and so interacts with the software. In addition, the software preferably provides a selectable word bank of emotions to identify and snapshot emotional state, and a range selector 0-10 to identify and snapshot intensity. These components preferably assist the user for the guided process, including maintaining focus on the displayed on-screen object by the user.

The system and method as shown herein are expected to provide a more effective therapeutic experience for treatment of PTSD and/or phobias in comparison to current treatment modalities, such as for example EMDR (Eye Movement Desensitization and Reprocessing).

According to at least some embodiments, there is provided a system for guiding a user during a treatment session for a mental health disorder, comprising a user computational device, the user computational device comprising a camera, a screen, a processor, a memory and a user interface, wherein said user interface is executed by said processor according to instructions stored in said memory, wherein eye movements of the user are tracked during the treatment session, and wherein the treatment session comprises a plurality of stages determined according to interactions of the user with said user interface and according to said tracked eye movements. Optionally, said user computational device further comprises a display for displaying information to the user, and wherein said memory further stores instructions for performing eye tracking and instructions for providing an eye stimulus by being displayed on said display, and wherein said processor executes said instructions for providing said eye stimulus such that said eye stimulus is displayed on said display to the user, and for tracking an eye of said user; wherein said instructions further comprise instructions for adjusting said eye stimulus according to said eye tracking. Optionally, said instructions further comprise instructions for moving said eye stimulus from left to right, and from right to left, according to a predetermined speed and for a predetermined period. Optionally, said instructions further comprise instructions for determining said predetermined period according to one or more of a physiological reaction of the user, tracking said eye of the user and an input request of the user through said user interface.

Optionally, said predetermined period comprises a plurality of repetitions of movements of said eye stimulus from left to right, and from right to left. Optionally, said instructions further comprise instructions for determining a degree of attentiveness of the user according to a biometric signal, and for adjusting moving said eye stimulus according to said degree of attentiveness. Optionally, said biometric signal comprises heart rate, the system further comprising a heart rate monitor, wherein said heart rate monitor transmits heart rate information to said user computational device. Optionally, said biometric signal comprises eye gaze, wherein said user computational device tracks eye gaze through said camera.

Optionally, said instructions further comprise instructions for determining whether said eye gaze tracking comprises high accuracy eye gaze tracking or low accuracy eye gaze tracking, and wherein said degree of attentiveness is calculated according to whether said eye gaze tracking comprises high accuracy eye gaze tracking or low accuracy eye gaze tracking, such that if said eye gaze tracking comprises high accuracy eye gaze tracking, said degree of attentiveness is determined according to a high degree of simultaneous overlap of locations of said eye gaze with locations of said eye stimulus; and alternatively wherein a lower extent of overlap of said locations is considered to calculate said degree of attentiveness.

Optionally, said instructions further comprise instructions for analyzing eye movements of the user according to one or more deep learning and/or machine learning algorithms. Optionally, said instructions further comprise instructions for analyzing biometric information from the user according to one or more deep learning and/or machine learning algorithms. Optionally, said one or more deep learning and/or machine learning algorithms comprise an algorithm selected from the group consisting of a DBN, a CNN and an RNN. Optionally, the user computational device further comprises a user input device, wherein the user interacts with said user interface through said user input device to perform said interactions with said user interface during the treatment session.

Optionally, the system further comprises a cloud computing platform, comprising a virtual machine, comprising a processor and a memory for storing a plurality of instructions; and a computer network, wherein said user computational device communicates with said cloud computing platform through said computer network; wherein said processor of said virtual machine executes said instructions on said memory to analyze at least biometric information of the user during a treatment session, and to return a results of said analysis to said user computational device for determining a course of said treatment session; wherein said biometric information is transmitted from a biometric measuring device directly to said cloud computing platform or alternatively is transmitted from said biometric measuring device to said user computational device, and from said user computational device to said cloud computing platform.

Optionally, said virtual machine analyses said biometric information from said biometric measuring device without input from said user computational device. Optionally, said virtual machine analyses said biometric information from said biometric measuring device in combination with input from said user computational device. Optionally, said biometric signal comprises heart rate, the system further comprising a heart rate monitor, wherein said cloud computing platform receives heart rate information directly or indirectly from said heart rate monitor. Optionally, said biometric signal comprises eye gaze, wherein said user computational device obtains eye gaze information from said camera, and wherein said cloud computing platform receives said eye gaze information from said user computational device. Optionally, said instructions further comprise instructions for determining whether said eye gaze tracking comprises high accuracy eye gaze tracking or low accuracy eye gaze tracking, and wherein said degree of attentiveness is calculated according to whether said eye gaze tracking comprises high accuracy eye gaze tracking or low accuracy eye gaze tracking, such that if said eye gaze tracking comprises high accuracy eye gaze tracking, said degree of attentiveness is determined according to a high degree of simultaneous overlap of locations of said eye gaze with locations of said eye stimulus; and alternatively wherein a lower extent of overlap of said locations is considered to calculate said degree of attentiveness.

Optionally, said instructions further comprise instructions for analyzing eye movements of the user according to one or more deep learning and/or machine learning algorithms. Optionally, said instructions further comprise instructions for analyzing biometric information from the user according to one or more deep learning and/or machine learning algorithms. Optionally, said one or more deep learning and/or machine learning algorithms comprise an algorithm selected from the group consisting of a DBN, a CNN and an RNN. Optionally, said instructions of said virtual machine comprise instructions for determining a determining a degree of attentiveness of the user according to said tracking of eye movements.

Optionally, the mental health disorder comprises PTSD (post traumatic stress disorder), a phobia or a disorder featuring aspects of PTSD and/or a phobia.

According to at least some embodiments, there is provided a method of treatment of a mental health disorder, comprising operating the system as described herein by a user, and adjusting said plurality of stages in the treatment session to treat the mental health disorder.

Optionally, the method further comprises a plurality of treatment stages to be performed in the treatment session, said treatment stages comprising a plurality of eye movements from left to right and from right to left as performed by the user, according to a plurality of movements of an eye stimulus from left to right and from left to right; wherein an attentiveness of the user at each stage to said movements of said eye stimulus and wherein a subsequent stage is not started until sufficient attentiveness of the user to a current stage is shown. Optionally, said treatment stages comprise at least Activation, wherein the user performs eye movements while considering a traumatic event; Externalization wherein the user performs eye movements while imagining themselves as a character outside of said traumatic event; and Deactivation, wherein the user performs eye movements while imagining such an event as non-traumatic. Optionally, said treatment stages further comprise Reorientation, wherein the user performs eye movements while re-imagining the event.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Unless otherwise defined, all technical and scientific terms used herein have the same meaning as commonly understood by one of ordinary skill in the art to which this invention belongs. The materials, methods, and examples provided herein are illustrative only and not intended to be limiting.

An algorithm as described herein may refer to any series of functions, steps, one or more methods or one or more processes, for example for performing data analysis.

Implementation of the apparatuses, devices, methods and systems of the present disclosure involve performing or completing certain selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be implemented as a number of software instructions being executed by a computer (e.g., a processor of the computer) using an operating system. In any case, selected steps of methods of at least some embodiments of the disclosure can be described as being performed by a processor, such as a computing platform for executing a plurality of instructions.

Software (e.g., an application, computer instructions) which is configured to perform (or cause to be performed) certain functionality may also be referred to as a “module” for performing that functionality, and also may be referred to a “processor” for performing such functionality. Thus, a processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions—which can be a set of instructions, an application, software—which are operable on a computational device (e.g., a processor) to cause the computational device to conduct and/or achieve one or more specific functionality.

Some embodiments are described with regard to a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 illustrates an example of a method 100 configured for facilitating one or more user(s) to participate in one of more treatment course(s)/session(s) in accordance with one or more implementations of the present invention;

FIG. 2 shows a non-limiting exemplary cloud computing platform for performing some aspects of the software systems and methods according to the present invention;

FIG. 3 shows a non-limiting exemplary implementation of the participant's computing device 220;

FIG. 4 shows a static treatment session 250 as a non-limiting flow;

FIG. 5 shows a non-limiting exemplary flow for completing a treatment;

FIG. 6 shows a session traversal logic 265 in an exemplary detailed flow;

FIG. 7 shows a non-limiting exemplary flow for the load instruction component 275;

FIG. 8 relates to a non-limiting exemplary flow for the load distress level component 280;

FIG. 9 relates to a non-limiting exemplary load emotion selector component shown flow 285;

FIG. 10 relates to a non-limiting exemplary load eye movement component at 290;

FIG. 11 relates to a non-limiting exemplary updated configuration of a participant computing device;

FIG. 12 shows a non-limiting exemplary flow of narrowband 330;

FIG. 13 shows a cloud computing platform 400 that features a dynamic treatment generation configuration 401;

FIG. 14 shows a non-limiting exemplary configuration of a therapy session engine;

FIG. 15 shows a non-limiting exemplary PTSD session treatment flow;

FIG. 16 shows an overall view of a non-limiting exemplary simple complete system;

FIG. 17 shows an additional non limiting exemplary system for performing the actions as described herein;

FIG. 18 shows a non-limiting exemplary system at a higher level, showing that a complete system 615 may be used for therapy as shown herein;

FIG. 19 shows a non-limiting exemplary complete system flow diagram;

FIGS. 20A and 20B relate to non-limiting exemplary systems for providing user signals as input to an artificial intelligence system with specific models employed, and then analyzing it to determine the effect of the treatment process on the user;

FIGS. 21A and 21B relate to non-limiting screens for reporting the type and intensity of emotions being experienced;

FIGS. 22A-22C relate to a non-limiting set of screens for recording a personal message;

FIGS. 23A-23E relate to a non-limiting set of screens for eye movement tracking; and

FIGS. 24A-24B show an exemplary eye tracking method in more detail.

### DESCRIPTION OF AT LEAST SOME EMBODIMENTS

The present invention, in at least some embodiments, provides a system and method including software and any associated hardware components, for use as a medical device to provide a therapeutic treatment where current clinical practices are less accessible and/or less desirable for the user. In one embodiment the therapeutic treatment is a psychological treatment, such as treatment of post-traumatic stress disorder (PTSD) and or phobia(s). In one embodiment, this is a user-directed treatment.

In at least some embodiment, the system of the present invention consists of a mobile app which can be installed on any device running Android and IOS. The system optionally features a web-interface which can be used from major browsers on any computer and/or a standalone software version which can be installed on a desktop, laptop or workstation computer.

In at least some embodiment, the system also includes wearables and eye-tracking sensors for the recording and collection of biometric data, which will enable further user engagement with the system.

In one example of the systems and methods of the present invention, various software components are provided in order to ensure user interaction, such as an animated ball or other client-customized stimulus that moves around the screen and that induces eye movements. The user tracks the visual stimulus and so interacts with the software. In addition, the software preferably provides a selectable word bank of emotions to identify and snapshot emotional state, and a range selector 0-5 to identify and snapshot intensity.

In one non-limiting example of the systems and methods of the present invention, a session is defined as a single set of interactions with a user, during which the software remains active. Even if the user does not finish all scripted stages or interactions, once the user deactivates or fails to interact with the software, the session is defined as being finished. These stages include the following:
Welcome/Introduction
Preparation
Activation
Externalization
Deactivation
Reorientation

In one example of the systems and methods of the present invention, as the user interacts with the software during each stage, preferably the user's interactions with the software are monitored. In addition, preferably the user's physiological state is monitored through a series of physiological measurements. These include eye tracking and heart rate measurements. Eye tracking is used to ensure that the user's iris moves as completely from left to right as is measurable. Without wishing to be bound by theory it is believed that the effectiveness of initiating the fight or flight response is higher when the rate of eye movement is faster than is normal, and the range of motion of the eye is broader rather than narrower. Therefore, in one embodiment of the systems and methods of the present invention, eye tracking is combined with on screen, visual and/or audio, prompts which induce the user to continue to follow the visual stimulus on the screen, and in certain embodiments, these prompts are varied according to the degree to which the user is maintaining eye tracking.

According to other embodiments of the present invention, the system and method include heart-rate measurements that are provided through a recording and transmission device. Monitoring heart rate during the session can be used as an indicator of stress/anxiety during the treatment. Such devices are known and may include wearables or other devices for heart rate measurements.

In certain embodiments, attentiveness is required of the user for the software to deliver the optimal results. The user is required to follow the visual stimulus to the greatest extent possible, and then to provide feedback on the user's state while doing so. Such feedback may then be correlated with physiological measurements such as eye tracking and heart rate measurements, to be certain that the user's description of their emotional state matches their physiological state. In certain embodiments, in an interactive session with the software alone, with the user moving through scripted stages while following the moving stimulus, this provides valuable information which may be used to determine the user's emotional state and also to adjust each stage according to feedback from the last stage or a plurality of last stages. For example, disjointed feedback or a failure to progress may indicate lack of attentiveness, and prompt a suggestion to return to the beginning or to stop the session. Additionally, in certain embodiments, over multiple such sessions, the software can adjust itself according to feedback from the individual user, alone or in comparison to feedback from other users. In one embodiment, this attentiveness by the user is then used to alter the trigger associated with a traumatic event to, instead, recall a non-threatening memory and response. In at least some embodiments, the system and methods of the present invention enables treatment which results in deactivating the neural network that previously triggered the fight or flight response that corresponds to the particular trauma stimuli.

In certain embodiments, the present invention incorporates multiple physiological measurements to determine a user's state and to assist the user. Furthermore, the present invention incorporates, in certain embodiments, staged sessions which incorporate functions from hypnosis, by having the user follow a visual stimulus while also providing suggested language prompts (as audio or visually, as text) to induce a therapeutic effect.

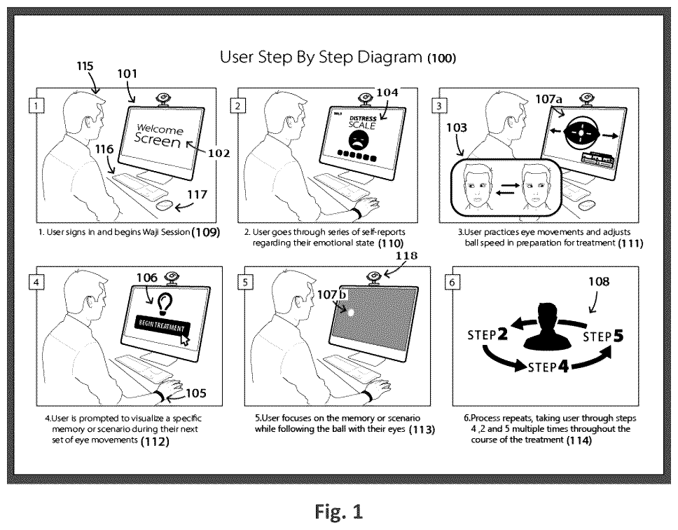

Turning now to the drawings, FIG. 1 illustrates an example of a method 100 configured for facilitating one or more user(s) to participate in one or more treatment course(s)/session(s) in accordance with one or more implementations of the present invention. The course(s)/session(s) may include, but is not limited to, one or more or any combination thereof of two or more steps shown in FIG. 1. In some implementations, the method 100 includes all of the steps in FIG. 1. FIG. 1 features a step by step diagram of how a user may interact with the non-limiting exemplary software according to the present invention. In one implementation, as shown in method 100 a user 115 interacts with a computer, which features a display 101, a keyboard 116 and a mouse 117. The user signs in and begins the software session 109 by looking at the welcome screen 102. The user then goes through a series of self reports regarding their emotional state at stage 110. This is shown through screen 104. Then the user conducts practice eye movements which adjust the ball speed in preparation for treatment at stage 111. In this stage the user has to follow the ball, which is displayed on the screen 107a with his/her eyes. The display screen 107a shows a ball moving back and forth, and the user's eyes will follow the ball and move back and forth at 103. Next, the user is prompted to visualize a specific memory or scenario during the next stage in stage 112. Here, the user may, for example, have their heart rate or heart pattern, measured with, for example, a wristband 105. The user then may optionally consider the screen 106 to determine for example, whether they should be beginning the treatment and also they should be visualizing their specific memory as they start. Next, the user focuses on the memory or scenario while tracking the ball with their eyes at 113. Here the ball is shown as 107b, and the user's camera, 118, preferably tracks the user's eyes. Then, at stage 114, the process repeats taking the user through the previous steps multiple times throughout the course of the treatment session/method, 108.

Without wishing to be limited by a single hypothesis, as the user looks left while tracking the eye stimulus with their eyes, the right side of their brain activates. When they look right, sensory information crosses the corpus callosum, which is the only primary neural pathway between both sides to activate the left side of the brain. Normally the two hemispheres do not communicate with each other much at all. It is known in the art that bilateral stimulation conduces whole brain synergistic function. The apparatus, system and method described herein employ this whole brain synergy, by giving users instructions and suggestions for each set of EMs (eye movements) in a very strategic way. Users are instructed to recreate their trauma only one time (in order to access more deeply the neural network extension that is associated with it) as opposed to the unlimited amounts associated with other therapies, and then to perform a sequence of steps (coupled with eye movements) to assist users to interface with their PTSD (their actual maladaptive automatic trauma response), in order to externalize and understand it, and imagine a different scenario that is not traumatic.

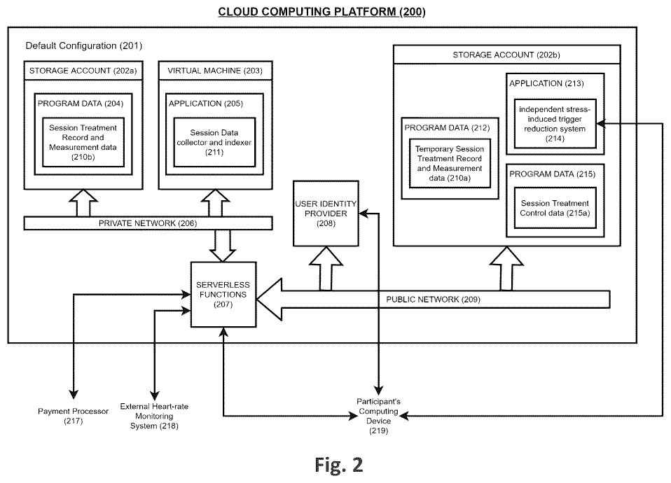

Turning now to FIG. 2, there is shown a non-limiting exemplary cloud computing platform for performing some aspects of the software systems and methods according to the present invention. As shown in the cloud computing platform 200, in an optional default configuration tool 201, there is provided a storage account 202a, which stores program data 204 and session treatment records and measurement data 210b. Optionally, this is operated by a virtual machine tool 203, which operates the application 205 including session data collector and indexer 211. This information may then communicate through a private network, 206, to apply other serverless functions 207, which, for example, may be provided as microservices. If the user is prompted to pay, the serverless functions 207 may include a link to a payment processor 217. The serverless functions 207 preferably also include a link to an external heart rate monitoring system 218, such as the wristband wearable shown in FIG. 1. The serverless functions also preferably communicate with the user's/Participant's computing device 219. This communicates with the user identity provider 208. The user is identified through user identity provider 208 so that the participants computing device 219 only connects with the proper user identity and is correctly identified also for the user's privacy. Serverless functions 207 may be communicated through a public network 209. In addition, public network 209 may support communication with user's/Participant's computing device 219. Also preferably in communication with public network 209 is a storage account 202b, which includes program data 212; a temporary session treatment record and measurement data 210a; application 213 which includes independent stress induced trigger reduction system 214; and program data 215 which includes the session treatment control data 215a. All of these communicate with the user's/participant's computing device 219. These two different storage accounts and information are preferably provided to support ease of access by the user and also local operation by the user on their local computing device.

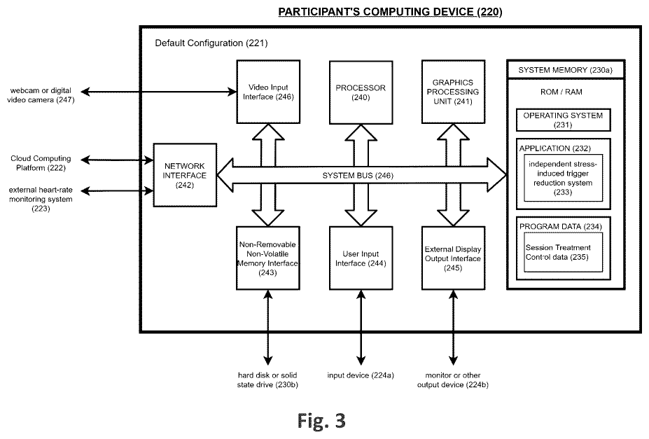

FIG. 3 shows a non-limiting exemplary implementation of the participant's computing device 220. A participant's computing device 220 preferably includes, in a default configuration 221, access to a webcam or digital camera 247 through a video import interface 246, access through a network interface 242 to a cloud computing platform 222 and to external heart rate monitoring system 223 as previously described. These preferably communicate with the system bus 246 which supports communication between these components and system memory 230a, which may also relate to storage of instructions for the operating system 231, application 232 which may include the independent stress induced trigger reduction and system 233 in a local instantiation. Program data 234 and session treatment control data 235, optionally in participant computing device 220, operates without reference to a server or to cloud computing, but alternatively may communicate with cloud computing platform 222 for example, to receive instructions, scripts and other information. Next, a non-removable non-volatile memory interface 243 preferably communicates with the hard disk or solid disk drive 230b. User input interface 244 communicates with an input device 224a, an external display output interface 245 communicates to the monitor other output device 224b.

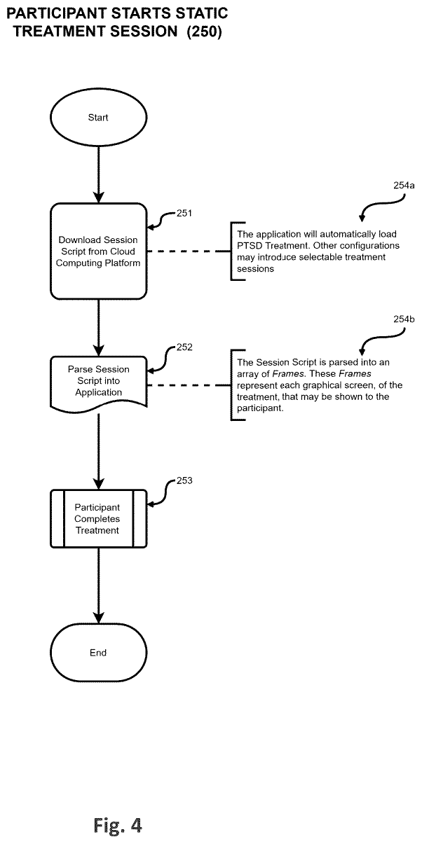

As shown, in a non-limiting exemplary flow chart, FIG. 4, there is provided a static treatment session 250. The session starts when a session script is downloaded from the cloud computing platform 251. The application automatically loads the PTSD treatment, other configurations may introduce selectable treatment sessions as shown in 254a. Next a session script is parsed into the application at 252. The session script is parsed into an array of frames; these frames represent each graphical screen of the treatment that may be shown to the participant in 254b and then the participant completes the treatment/session in 253 after which the session ends.

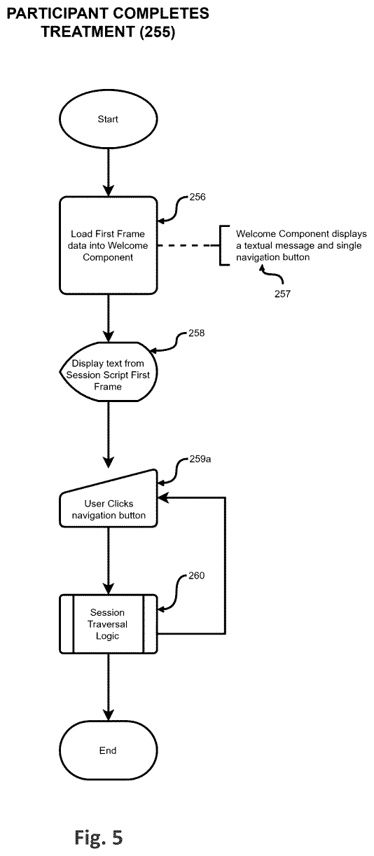

FIG. 5 shows a non-limiting exemplary flow for completing a treatment. As shown, the participant completes the treatment in flow 255. First, the first frame data is loaded into the welcome component 256. The welcome component displays a textual message and single navigation button at 257. The text from the session script first frame is displayed at 258. The user clicks the navigation button at 259a. The session traversal logic is performed at 260 and the user continues to click navigation buttons at 259a. These steps are preferably repeated until the session is complete.

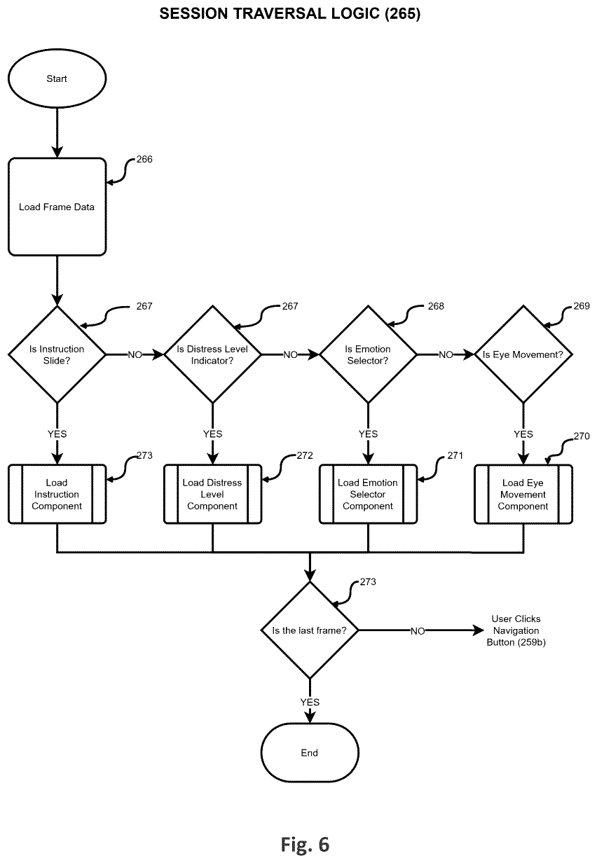

In FIG. 6, a session traversal logic 265 is shown in an exemplary detailed flow. The session traversal logic 265 begins by loading the frame data at 266. Then it is determined if it is an instruction slide at 267, if yes then the instruction component is loaded at 273. If not, then it is determined whether there's a distress level indicator at 267, if so the distress level component is loaded at 272. Otherwise, if it is an emotion selector 268, the emotion selector component is loaded at 271. If it is eye movement at 269, then the eye movement component is loaded 270. When the correct component has been loaded, then this process is repeated until it is determined if in fact, it is the last frame at 273. If that's the case the process ends, otherwise the user is required to click a navigation button at 259b or otherwise participate.

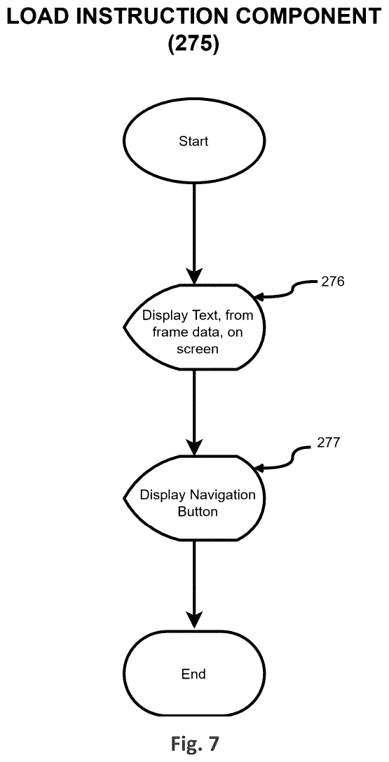

FIG. 7 shows a non-limiting exemplary flow for the load instruction component 275. The process preferably starts at 276 when display text from the frame data is shown on the screen. The navigation button is then displayed 277 and the flow ends.

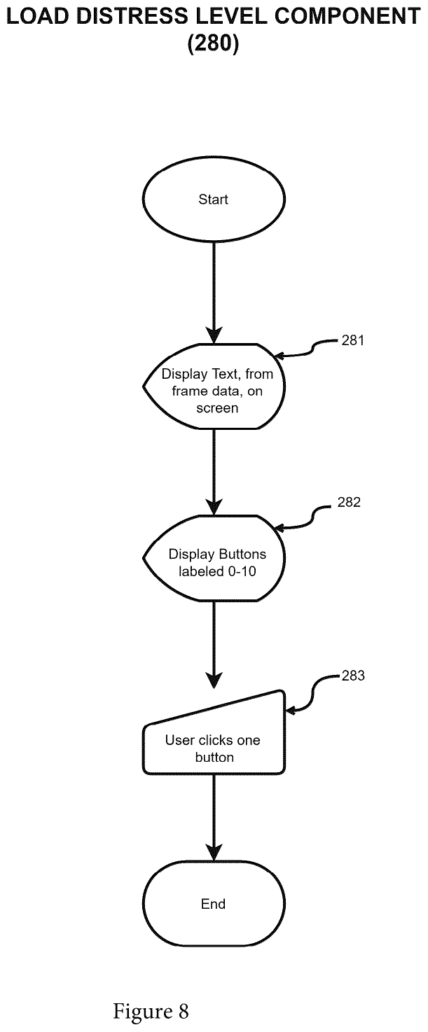

FIG. 8 relates to a non-limiting exemplary flow for the load distress level component 280. This process preferably starts by displaying text from the frame data on the screen at 281. Buttons may then be displayed labeled 0-10 at 282 to indicate the stress or some other type of labeling may be provided or display may be provided. The user then selects a distress level for example by clicking a button at 283 and the flow ends.

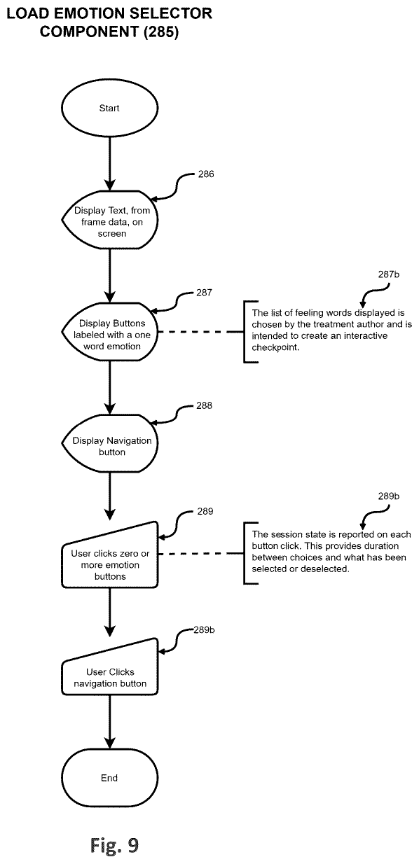

FIG. 9 relates to a non-limiting exemplary load emotion selector component shown flow 285. The flow preferably starts at 286 when text is displayed from the frame data on the screen. Then optionally, buttons or some other type of GUI gadget or widget or preferably displayed with a one word emotion at 287. The list of these emotional words displayed may be chosen by the treatment author and isn't intended to create an interactive check point at 287b. The author may for example be a therapist. Next, the navigation button is displayed at 288, and the user clicks zero or more emotion buttons at 289 or other GUI gadgets or otherwise vindicates an emotion. The session state is reported; optionally for each button click, such state reporting provides duration between choices and what has been selected or deselected at 289b. The duration between choices may be important for example to indicate emotional distress, or the need for further consideration by the user. At 289b the user clicks the navigation process and this flow ends.

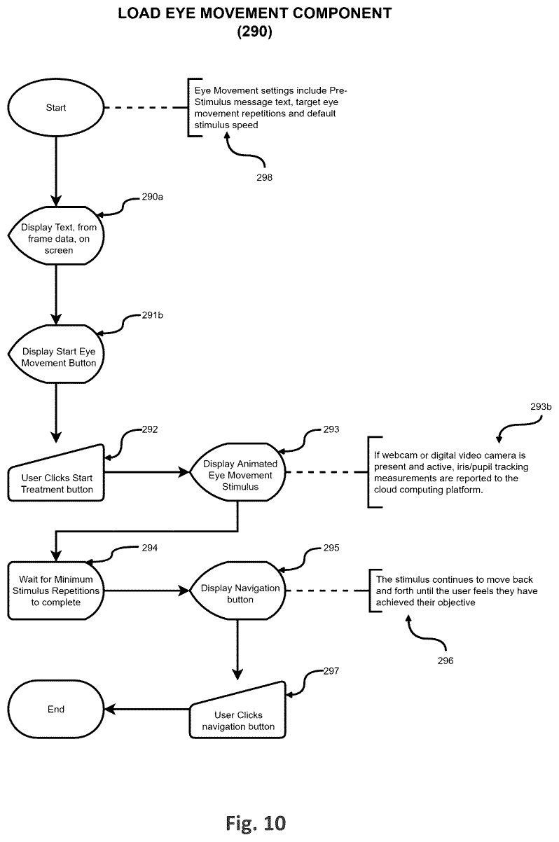

FIG. 10 relates to a non-limiting exemplary load eye movement component at 290. The eye movement settings include the pre-stimulus message text target eye movement repetitions and default stimulus speed at 298. Next at 290a, the text is displayed from the frame data on the screen. At 291b the display of the start eye movement button is provided. The user clicks the start treatment button at 292. An animated eye movement stimulus is then perfectly displayed at 293. If the web camera or digital video camera is present and active, iris/pupil tracking measurements may be reported to the cloud computing platform at 293b. Next, the process waits for a minimum stimulus repetitions to complete at 294, the navigation button is displayed at 295. The stimulus continues to move back and forth until the user feels they've achieved their objective at 296. The user then clicks the navigation button at 297 and the flow ends.

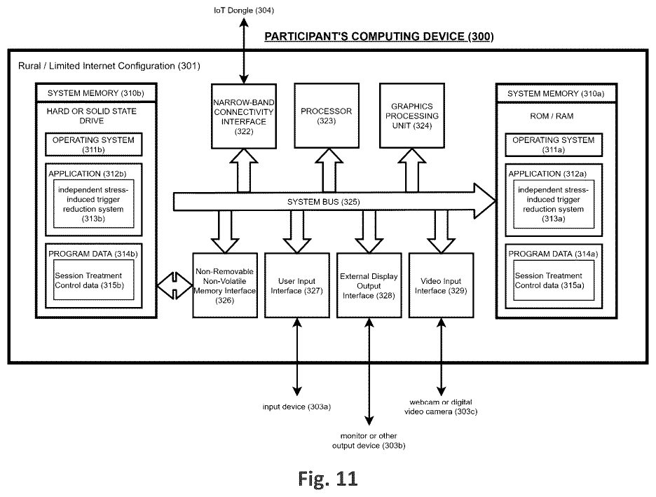

FIG. 11 relates to a non-limiting exemplary updated configuration of a participant computing device. A participant computing device 300 may optionally not feature a direct connection to cloud computing or may be able to operate the process independently of cloud computing, for example, in a rural limited internet configuration 301. An IoT Dongle 304 may optionally provide narrowband connectivity interface 322 if in fact connectivity is possible. A processor 323 and graphics processing unit 324 communicate with a system bus 325. Non-removable non-volatile memory interface 326 preferably communicates with a system bus 325 as does user input interface 327 external display output interface 328 and video input interface 329. User input interface 327 preferably communicates with an input device 303a which may for example be a mouse or keyboard or touchscreen. External display output interface 328 preferably communicates with the monitor other output device 303b and video input interface 329 preferably communicates with the webcam or digital video camera 303c. System memory 310a preferably hosts an operating system 311a including application 312a which includes an independent stress induced trigger reduction system 313a, program data 314a, which includes session treatment control data 315a. System memory 310b preferably includes a solid or hard state drive, which operates an operating system 311b this also preferably stores an application 312b which again includes an independent stress induced trigger reduction system 313b and program data 314b which includes a session treatment control data 315b.

Also optionally, memory 310B is configured for storing a defined native instruction set of codes. Processor 323 is configured to perform a defined set of basic operations in response to receiving a corresponding basic instruction selected from the defined native instruction set of codes stored in memory 310B. For example and without limitation, memory 310B may store a first set of machine codes selected from the native instruction set for receiving session treatment data (for example with regard to eye tracking) and a second set of machine codes selected from the native instruction set for indicating whether the next screen should be displayed to the user as described herein.

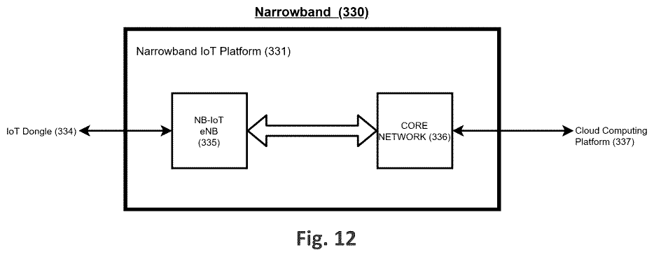

FIG. 12 shows a non-limiting exemplary flow of narrowband 330. This flow can assist with a computational device that has limited access to a cloud computing program. For example, to upload data or to download scripts, it can use an IoT dongle 334. This is connected to a narrow band IoT platform 331, and NB-IoTeNB 335 communicates to the core network 336 and then that communicates with Cloud computing platform 337.

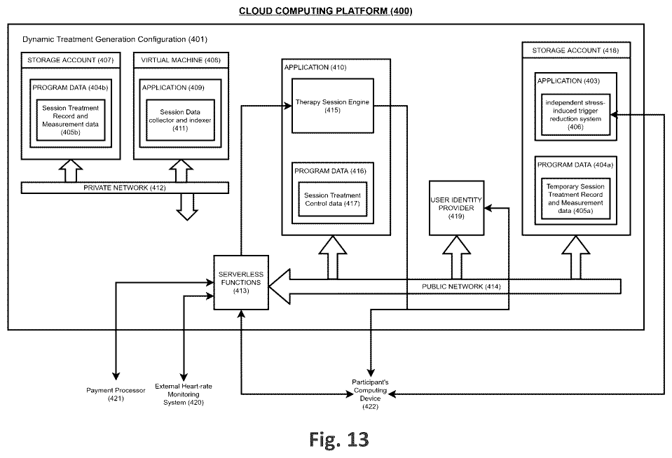

Next as shown in FIG. 13, a cloud computing platform 400 features a dynamic treatment generation configuration 401. In this configuration, the same modules are included as before, but in this case program data 404a includes temporary session treatment record and management data 405a. The application 403 includes an independent stress induced trigger reduction system 406. This information also relates to application 409, which includes a session data collector and indexer 411. Program data 404b includes a session of treatment record and measurement data 405b. Many components in cloud computing platform 400 function as previously described.

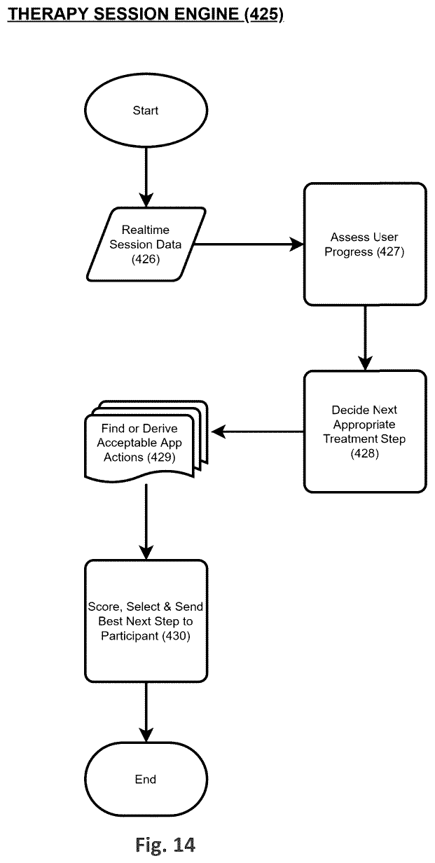

A therapy session engine is shown in FIG. 14 which is a non-limiting exemplary configuration. As shown, a therapy session engine 425 receives real time session data 426 and assesses the user progress in 427. For example, whether or not the user is actually tracking the ball on the screen with his or her eyes, whether or not the user is responding fully and frankly, or if the user is focused and also optionally whether the user's treatment is progressing. The engine then decides the next appropriate treatment step at 428 and finds or derives acceptable app actions at 429. Next the score select and send best next step to participant is performed at 430 so that the engine can send the information to assist the participant in the next step to be performed through the app.

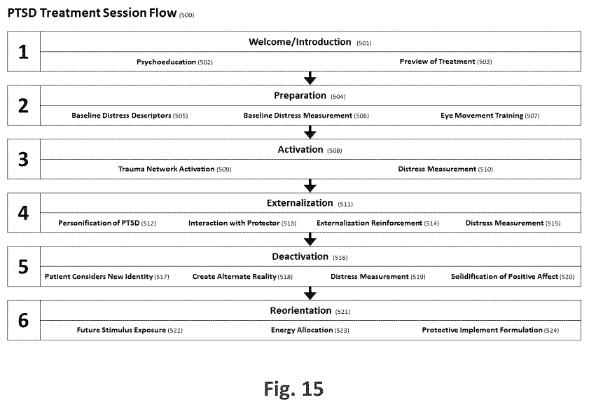

FIG. 15 shows a non-limiting exemplary PTSD session treatment flow. The flow stages may be summarized as follows:
Welcome/Introduction
Preparation
Activation
Externalization
Deactivation
Reorientation

As shown in a flow 500, the first screen begins with a welcome and introduction at 501, which includes psychoeducation 502 and preview of treatment at 503. In the next step, the user is prepared at 504 for treatment. This step of preparation may include, for example, baseline distress descriptors 505, baseline distress measurement 506, and eye movement training 507. Next activation is performed at 508. This step of activation may include trauma network activation 509 and distress measurement 510. Then externalization is performed at 511. The step of externalization may include the personification of the PTSD at 512. The protector interaction occurs at 513. The externalization reinforcement occurs at 514; this step may include distress measurement at 515. The activation is performed at 516. The step of activation may include the patient considering a new identity at 517, creating an alternative reality at 518, the stress measurement at 519, and the solidification of positive effect at 520. Next reorientation is performed at 521. The step of reorientation may include a future stimulus exposure at 522, energy allocation of 523 and protective implement formulation at 524.

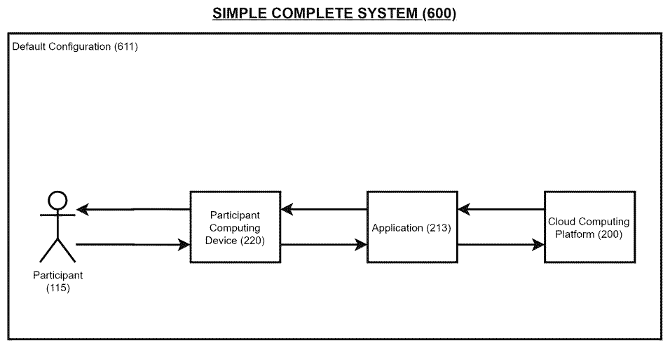

Turning now to FIG. 16, there is shown an overall view of a non-limiting exemplary simple complete system. A system 600 features a default configuration 611 with a participant 115 controlling participant computing device 220 as previously described. Participant computing device 220 runs application 213, which in turn receives information and also passes data to cloud computing platform 200. In this flow, participant 115 views a light that is moving on the screen or participant computing device 220 or view some other type of moving stimulus. As participant 115 tracks a stimulus with their eyes, then application 213 engages in a therapy session. For example, providing additional instructions for the user, participant 115, including but not limited to providing feedback, selecting an emotional state and performing other actions.

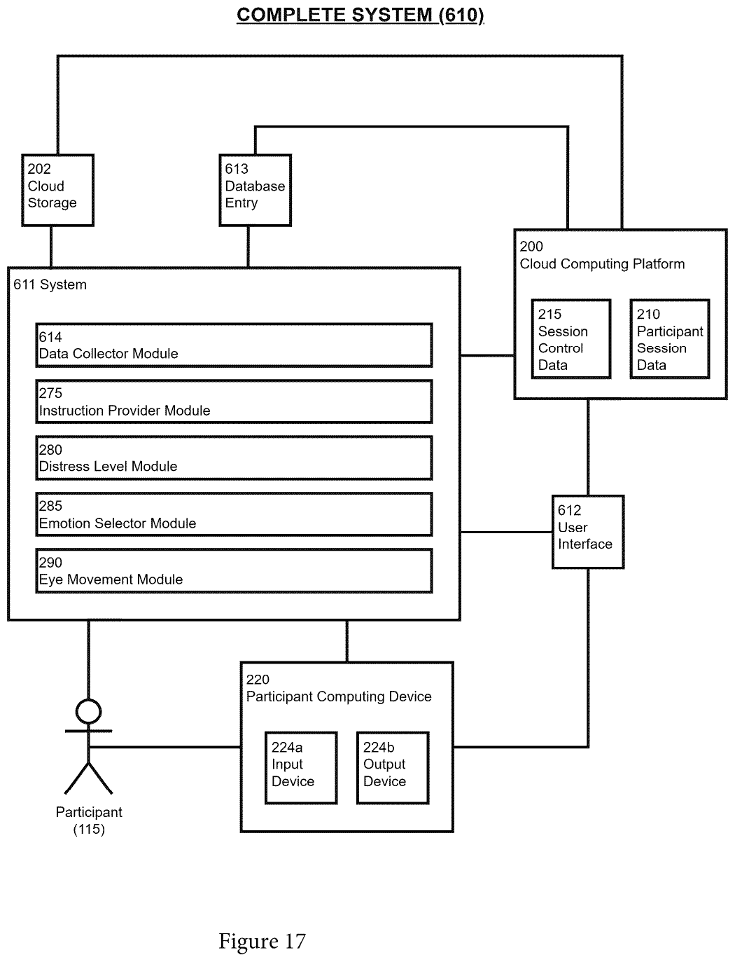

FIG. 17 shows an additional non limiting exemplary system for performing the actions as described herein. As shown a system 610 features cloud storage 202 and database entry 613. The information stored in cloud storage may, for example, relate to data provided by the users and scripts for being performed, for example, for the previously described session. Cloud computing platform 200 provides session control data 215 participants session data 210 as previously described. System 611 includes a data collector module 614 for collecting data. The user data is collected and isn't analyzed. For example, the user may or may not be following the stimulus such as a light with their eyes on the screen. The user also may or may not be following a particular script. If the script is not followed or other actions are not taken, or conversely if the actions are taken, but perhaps showing a spike in user pulse or other information, this information is collected by data collector module 614. Instructor provider module 275 provides instructions and distress level module 280 measures distress. Emotional section module 285 helps the user to select emotions or may provide emotional cues. An eye movement module 290 tracks the movement of the users eye, for example, for the previously described iris or pupil tracking. User interface 612 allows the user to control the user application, including but not limited to, changing the speed of the stimulus such as a light and also uploading a particular script and giving permission for the user data to be provided to the system. All of this is performed through participant computing device 220, which includes an input device 224a, and an output device 224b. The input device may, for example, be a mouse or keyboard and the output device may, for example, be a display screen. Participant 115 controls system 611, user interface 612 and participant computing device 220 and also determines the data that is collected and that may be shared with additional components within the system.

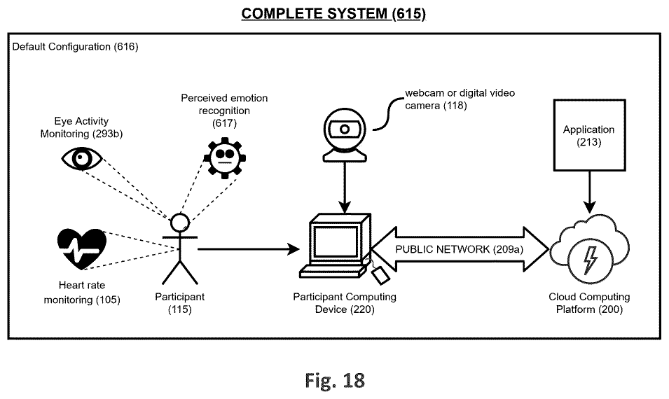

FIG. 18 shows a non-limiting exemplary system at a higher level, showing that a complete system 615 may be used for therapy as shown herein. A default configuration 616 provides information, such as, eye monitoring activity 293b. A perceived emotion recognition model 617 and a heart rate monitoring 105 which, for example, maybe through a wearable such as a watch. Participant 115 performs eye activity which is then monitored and gives information with regard to emotion or thirdly has this information gathered from biometrics, and provides metrics such as heart rate monitoring. The session is controlled through participant computing device 220, which may be connected for example, through a public network 209a to a cloud computing platform 200. Application 213 may be operated on participant computing device 220 or maybe run entirely through cloud computing platform 200. A web camera digital video camera 118 as preferably provided with participant computing device 220 to enable the eyes of the user to be tracked.

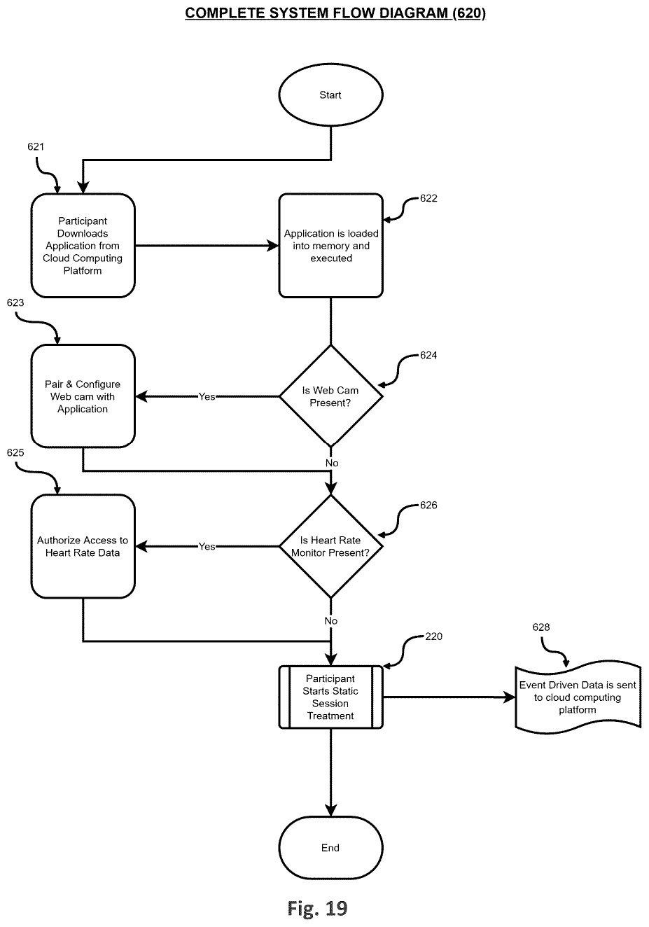

FIG. 19 shows a non-limiting exemplary complete system flow diagram. As shown in the system 620 this flow starts at the start. Next, the participant downloads the application from the cloud computing platform at 620. Alternatively instead of a download the application may be run through the cloud computing platform. Next the application is loaded into memory and executed at 622. If a webcam is present and shown to be active, then it is paired and configured with the application at 623 so that the user's eyes can be tracked. If not then, or alternatively, after such pairing configuration, then a heart rate monitor is detected at 626. If the heart rate monitor is present, such as, for example through a wearable which may send data directly to the system at 625 the user authorizes access to the heart rate data. The process continues in any case with a static session treatment at 220. Then event driven data is sent to the cloud computing platform at 628 and the session then ends.

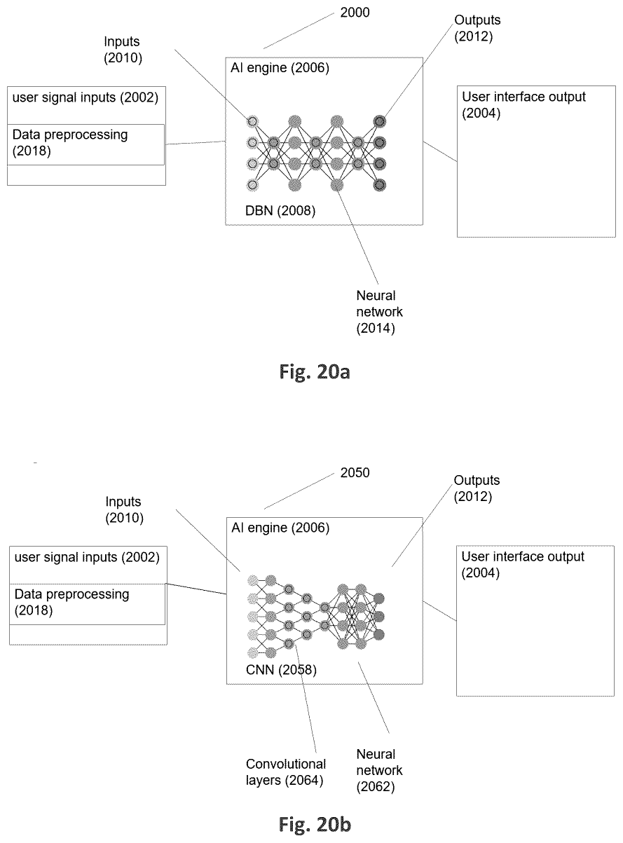

FIGS. 20A and 20B relate to non-limiting exemplary systems for providing user signals as input to an artificial intelligence system with specific models employed, and then analyzing it to determine the effect of the treatment process on the user. Such user signals may include eye tracking and determination of eye gaze, as well as heart rate and other physiological measurements. After analyzing the user signals, preferably the engine adjusts the user software application as previously described. Such artificial intelligence systems may for example be incorporated into the previously described application 213 and/or independent stress induced trigger reduction system 214 of FIG. 2.

Turning now to FIG. 20A and as shown in a system 2000, user signals input 2002 provides voice data inputs that preferably are also analyzed with the data preprocessing functions in 2018. The pre-processed information may for example include the previously described eye tracking. This data is then fed into an AI engine in 2006 and user interface output 2004 is provided by the AI engine. The user interface output 2004 preferably includes information for controlling the previously described user application, for example by adjusting the script.

In this non-limiting example, AI engine 2006 comprises a DBN (deep belief network) 2008. DBN 2008 features input neurons 2010, processing through neural network 2014 and then outputs 2012. A DBN is a type of neural network composed of multiple layers of latent variables (“hidden units”), with connections between the layers but not between units within each layer.

FIG. 20B relates to a non-limiting exemplary system 2050 with similar or the same components as FIG. 20A, except for the neural network model. In this case, a neural network 2062 includes convolutional layers 2064, neural network 2062, and outputs 2012. This particular model is embodied in a CNN (convolutional neural network) 2058, which is a different model than that shown in FIG. 20A.

A CNN is a type of neural network that features additional separate convolutional layers for feature extraction, in addition to the neural network layers for classification/identification. Overall, the layers are organized in 3 dimensions: width, height and depth. Further, the neurons in one layer do not connect to all the neurons in the next layer but only to a small region of it. Lastly, the final output will be reduced to a single vector of probability scores, organized along the depth dimension. It is often used for audio and image data analysis, but has recently been also used for natural language processing (NLP; see for example Yin et al, Comparative Study of CNN and RNN for Natural Language Processing, arXiv:1702.01923v1 [cs.CL] 7 Feb. 2017).

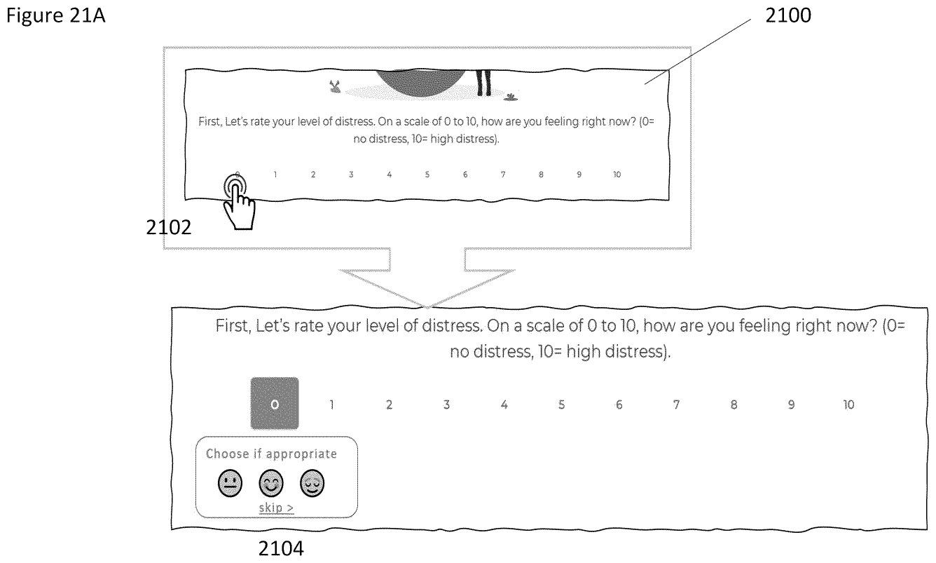

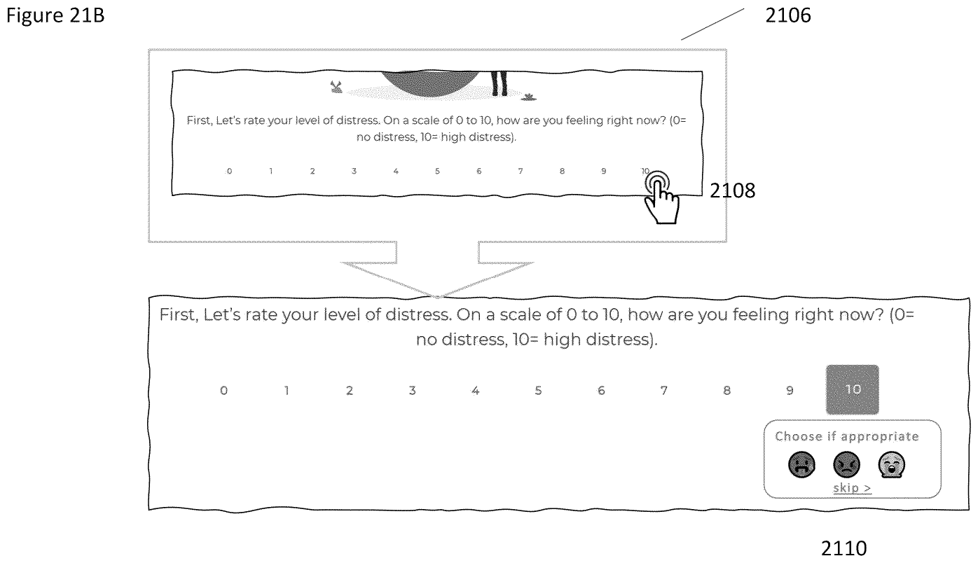

FIGS. 21A and 21B relate to non-limiting screens for reporting the type and intensity of emotions being experienced. In order to assist the user to more accurately describe their current emotional state during treatment, the standard 0 to 10 Stress Selector approach is extended by prompting the user, after making their initial 0 to 10 selection, they are prompted to further qualify the type, or flavor, of their feelings. This is expressed in the current embodiment of the treatment with a curated selection of emojis that correspond to their intensity selection. The visual representation of feelings via a Visual Analog Scale assists the user in accurately understanding and expressing their own emotional state.

Via follow-up interviews and other feedback received from patients, without wishing to be limited by a single hypothesis, though the standard measurement for “gauging stress level” is a 0-10 scale, this only measures the intensity of feeling and does not classify or qualify whether the stress is from rage or despondent sadness. This difference is particularly significant in participants that make a selection of 8-10; while those that select lower scores have generally categorized their determination for their selection between “not feeling stressed” and “being in a good mood”.

These significant nuances are success factors that inform the system of the user's mindset, intent and progress within the treatment. These success factors are used in the following ways, both in treatment and throughout the course of the user's mastery of their stress: determining whether their emotional state aligns with others who have had success with the treatment; and inferring how well the user is benefitting from each stage as that user progresses through each stage.

In response to the information provided herein, the system may take one or more of the following actions: adjusting the language to provide better targeted, or preferred, instruction and encouragement; repeating, retrying or skipping certain steps.

As shown in FIG. 21A, which is a mock-up of a first screen 2100, the user is asked to select which number most closely represents the feeling of the user at this time, where 0=no distress and 10=high distress. The user is represented as schematically selecting 0 at 2102. In the next screen, at 2104, the user is then asked to select which type of emotion most closely represents how the user is feeling.

FIG. 21B is a mock-up of another screen 2106, in which the user is asked to select which number most closely represents the feeling of the user at this time. This user may be a different user or the same user as for FIG. 21A, but at a different time point. In this case, the user is represented as schematically selecting 10 at 2108. In the next screen, at 2110, the user is then asked to select which type of emotion most closely represents how the user is feeling.

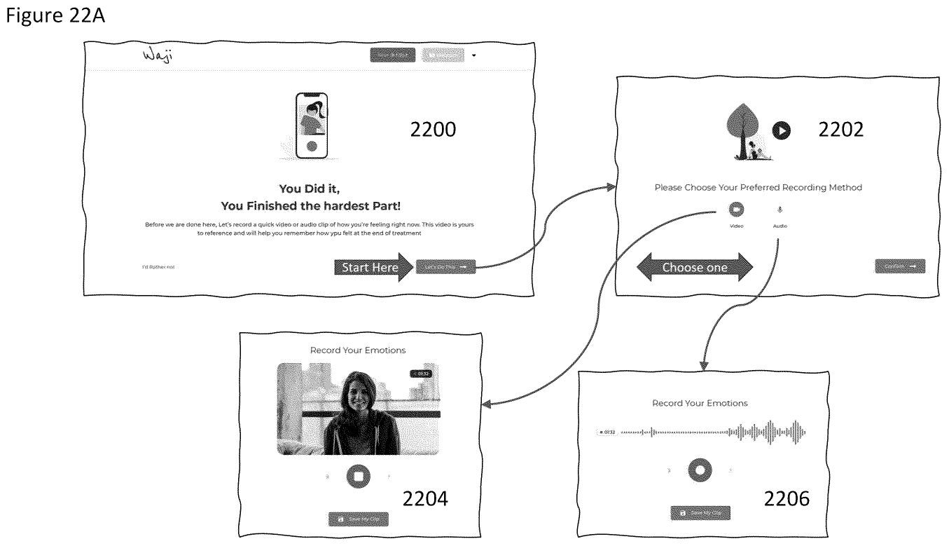

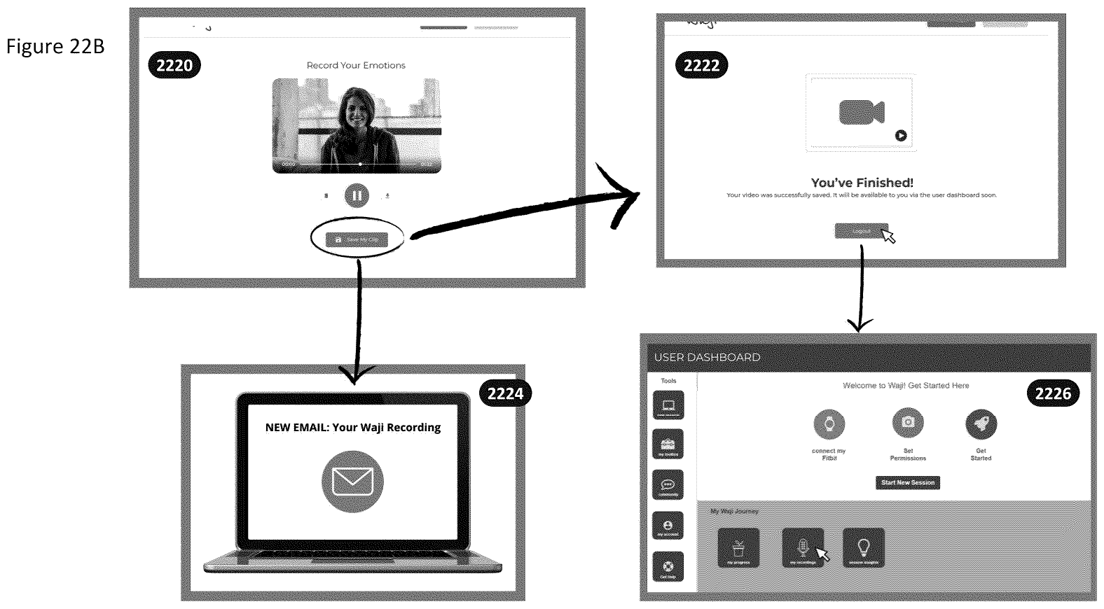

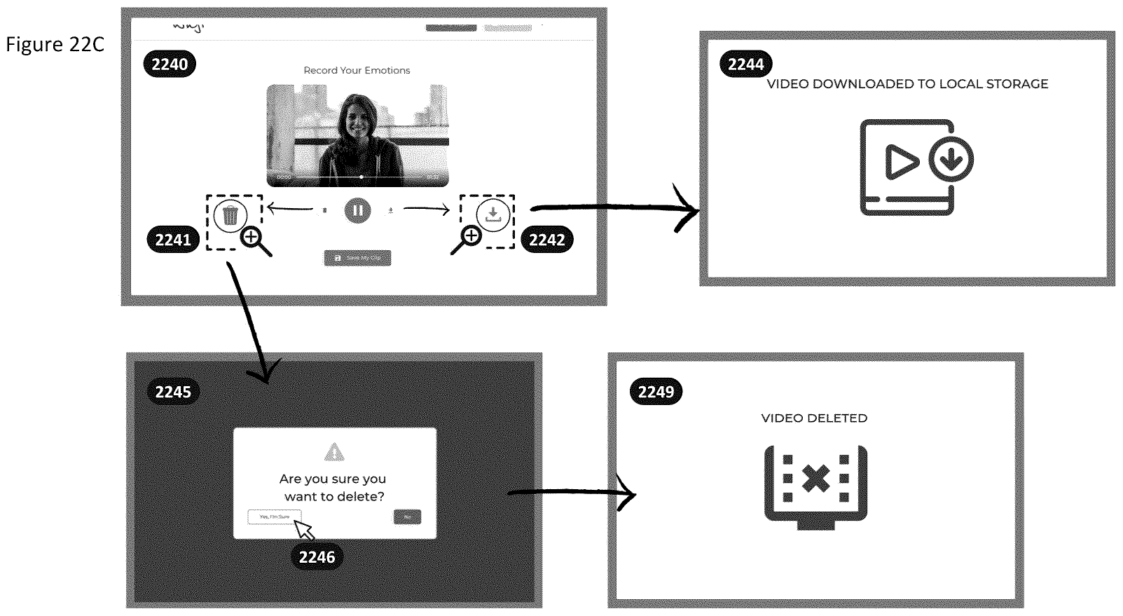

FIGS. 22A-22C relate to a non-limiting set of screens for recording a personal message. In many users, a strong feeling of relief is felt, in addition to a reduction in their triggered stress responses, following a successful round of treatment performed according to the present invention as described herein. While the stress triggers typically maintain their reduction, the sense of relief can fade over time. This fading of relief can cause the user to either forget what had actually healed them or sometimes to question the effectiveness of the treatment, creating a new cause of anxiety.

In order to aid the user in reliving/retrieving the experience of relief they felt following their successful round of treatment performed according to the present invention as described herein, the user may create an “Anchor” memoriam to capture the experience in a personally meaningful way for future use. An Anchor may be created after any successful treatment as described herein. In the current embodiment the Anchor may be captured in the following forms: Letter/Journal Entry; Audio Recording; or combined Audio/Video Recording.

The system can later provide/reproduce this Anchor on-demand so that the user is able to trust their own report that things are better. This experience is usually the last time they question whether they are affected by the trauma symptoms treated in the session(s) associated with that Anchor.

The system and method as described herein is primarily self-administered without a clinician's support. The Anchor serves as a superior replacement, as a preserved message to oneself is a arguably more genuine reminder to oneself than an ad-hoc call with a clinician.

As shown with regard to a schematic series of screens in FIG. 22A, in a first screen 2200, the user has successfully finished one treatment step or a plurality of such steps. Screen 2200 encourages the user to make an Anchor message, for replay later on, to support the user. Screen 2202 asks the user to select a recording method. In screen 2204, the user may record a message with video. In screen 2206, the user may record a message with audio only.

FIG. 22B shows a schematic series of screens for recording a video message. The user records the video message at 2220. When they are done, the video message is emailed to the user, whether as an attachment or a link, at 2224. A congratulations message screen is shown at 2222. The user is given more choices of further actions at 2226, for example to review previously recorded messages or other types of messages, such as audio messages for example.

FIG. 22C shows a schematic series of screens for downloading and/or deleting a video message. At 2240, the user may select to delete a video message, or to download it for local or other storage. At 2245, if the user selects deleting the video message, they need to confirm first, after which it is deleted. At 2249, confirmation of deletion is provided. At 2244, the video is downloaded to a local or other storage if the user has made that selection.

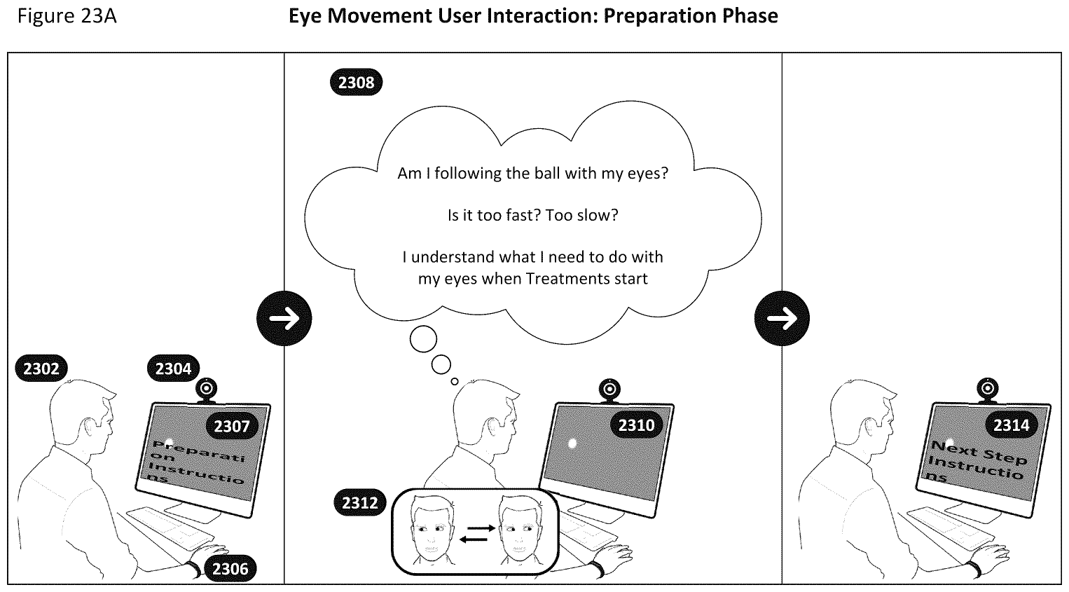

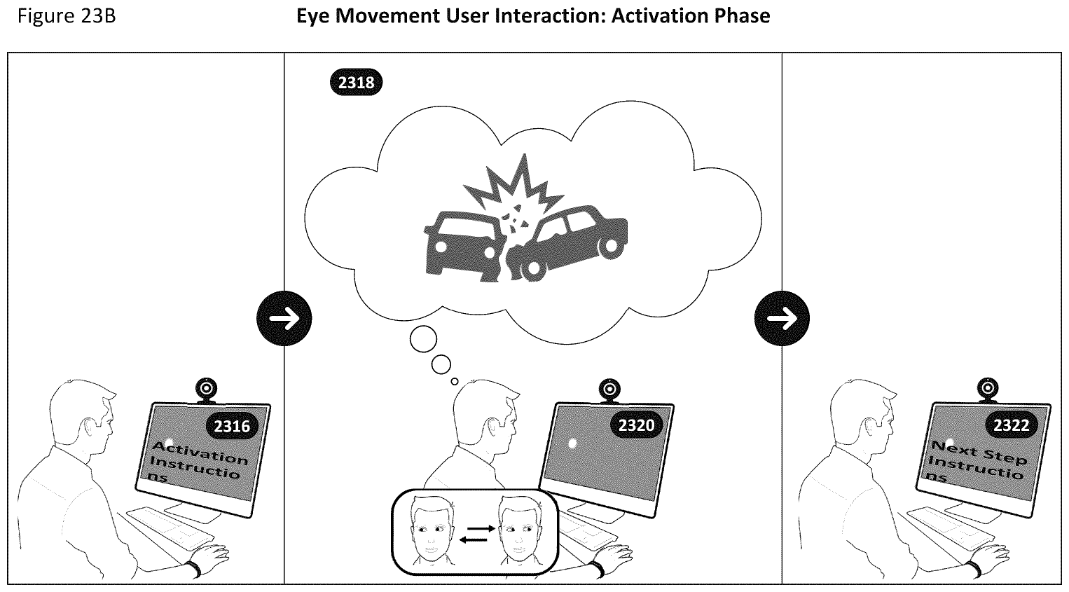

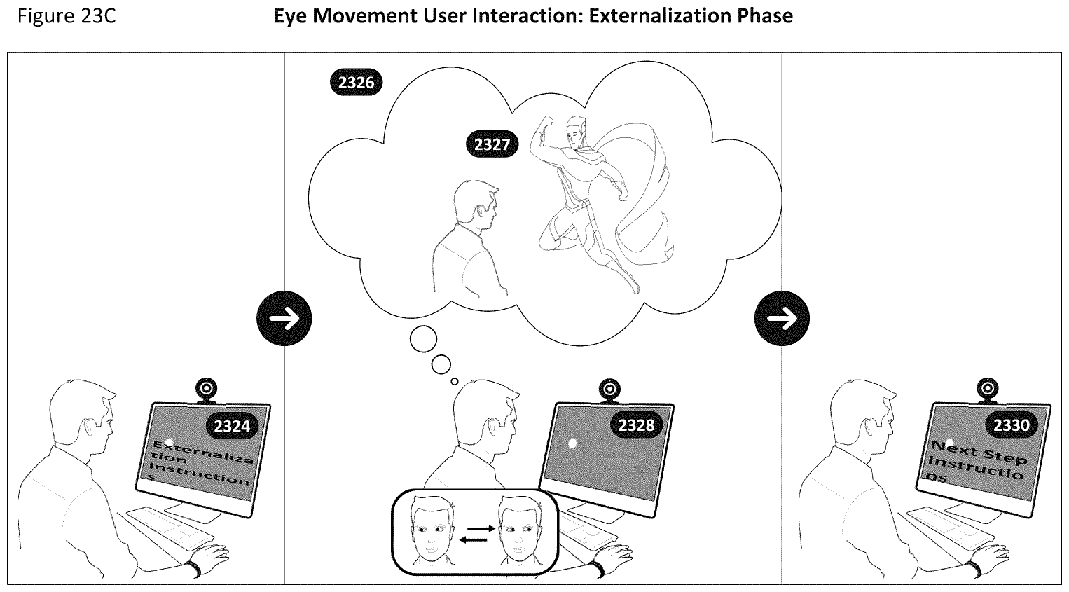

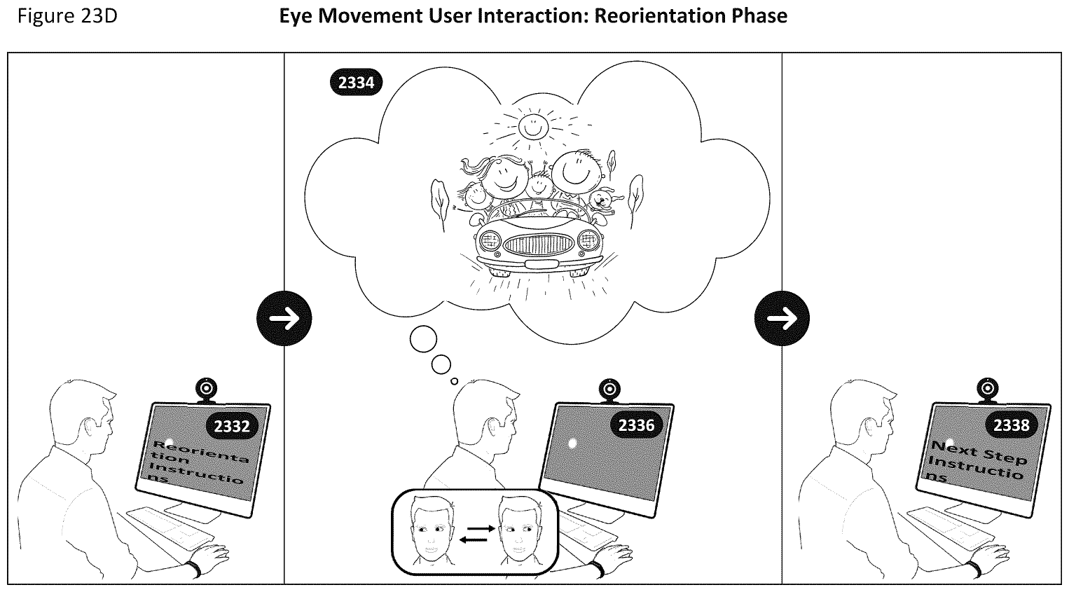

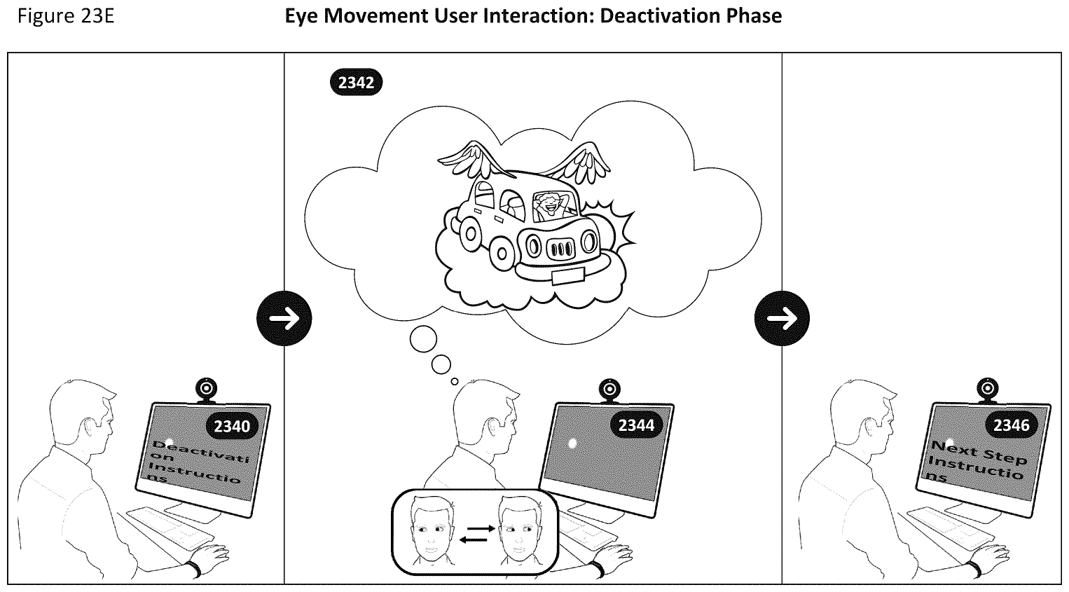

FIGS. 23A-23E relate to a non-limiting set of screens for eye movement tracking. As an overall description of the method shown herein, a user is preferably given instructions and suggestions for each set of EMs (eye movements) in a very strategic way. Each user preferably recreates their trauma only one time (in order to access more deeply the neural network extension that is associated with it) as opposed to the unlimited amounts associated with other therapies. Next the user interfaces with their PTSD (their actual maladaptive automatic trauma response) in order to externalize and understand it, and then to imagine a different scenario that is not traumatic.

FIG. 23A shows an initial screen for Eye Movement User Interactions in the Preparation Phase. A user 2302 operates a computer featuring a display screen 2307, with a webcam 2304 and a keyboard, mouse or keyboard and mouse 2306. In the next (middle) panel, user 2302 follows the instructions displayed at 2310, to follow a symbol (which may be a ball, image, cursor, and so forth) moving along display screen 2307 with their eyes. An inset panel 2312 shows user 2302 following the symbol with their eyes through their eye movements. Such eye movements may be performed a plurality of times, which may be determined by the system and/or which may be determined according to the reaction of user 2302, for each of FIGS. 23A-23F as shown herein. User 2302 is shown as performing such eye movements at 2308. At the right most panel, a display of next instructions is shown at 2314. Moving between such stages (screens) may be determined by the system and/or by user 2302, for example according to the reaction of user 2302 to one or more prompts, for each of FIGS. 23A-23F as shown herein.

FIG. 23B shows a next screen for Eye Movement User Interaction: Activation Phase. The user 2302 views a screen display of activation instructions 2316. In the middle panel, user 2302 thinks about a traumatic event, shown representationally at 2318, while following the symbol with their eyes through display 2320. Again the inset panel shows user 2302 following the symbol with their eyes through their eye movements as they think about this traumatic event. At the right most panel, a display of next instructions is shown at 2322.

FIG. 23C shows a next screen for Eye Movement User Interaction: Externalization Phase. The user 2302 views a screen display of externalization instructions 2328. In the middle panel, user 2302 thinks about the same traumatic event again but altered to externalize the event to user 2302, shown representationally at 2326, while following the symbol with their eyes through display 2328. Eye movements assist the user in drawing from his/her imagination a metaphoric character that represents his/her trauma reaction, i.e., symptoms of PTSD. Users are able to disidentify from their symptoms and also realize that they are not inherently bad. Again the inset panel shows user 2302 following the symbol with their eyes through their eye movements as they think about this traumatic event. At the right most panel, a display of next instructions is shown at 2330.

FIG. 23D shows a next screen for Eye Movement User Interaction: Reorientation Phase. The user 2302 views a screen display of reorientation instructions 2332. In the middle panel, user 2302 thinks about the setting of the same traumatic event again but re-imagined as a happy event, shown representationally at 2334, while following the symbol with their eyes through display 2336. As another non-limiting example, user 2302 visualises exposure to circumstances similar to that of the traumatic event, as a form of exposure therapy. Again the inset panel shows user 2302 following the symbol with their eyes through their eye movements as they think about this new version of the event. At the right most panel, a display of next instructions is shown at 2338.

FIG. 23E shows a next screen for Eye Movement User Interaction: Deactivation Phase. The user 2302 views a screen display of deactivation instructions 2340. In the middle panel, user 2302 generalizes the event to one with positive emotional content, shown representationally at 2342, while following the symbol with their eyes through display 2344. For example, user 2302 may be encouraged to create an alternate reality in which the traumatic event did not happen or was somehow different. Eye movements encourage escape from logical constraints and are conducive to out of the box cognitive operations. Again the inset panel shows user 2302 following the symbol with their eyes through their eye movements as they think about this new version of the event. At the right most panel, a display of next instructions is shown at 2346.

The process shown in FIGS. 23A-23E may be repeated at least once or a plurality of times.

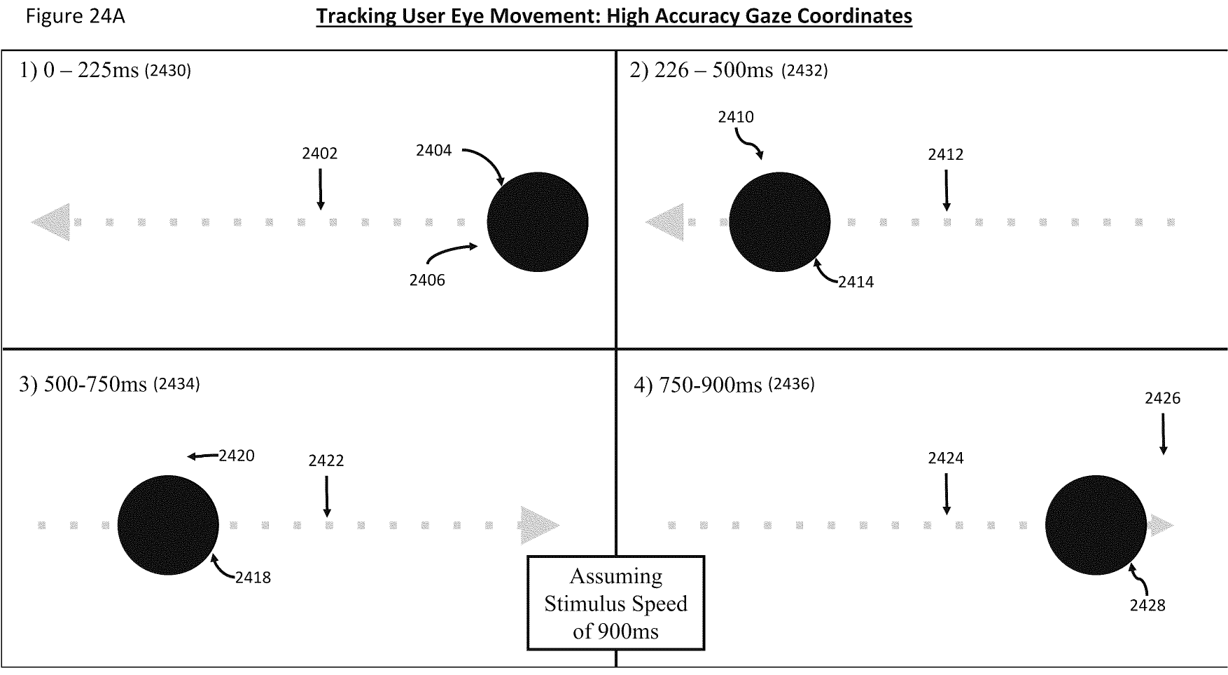

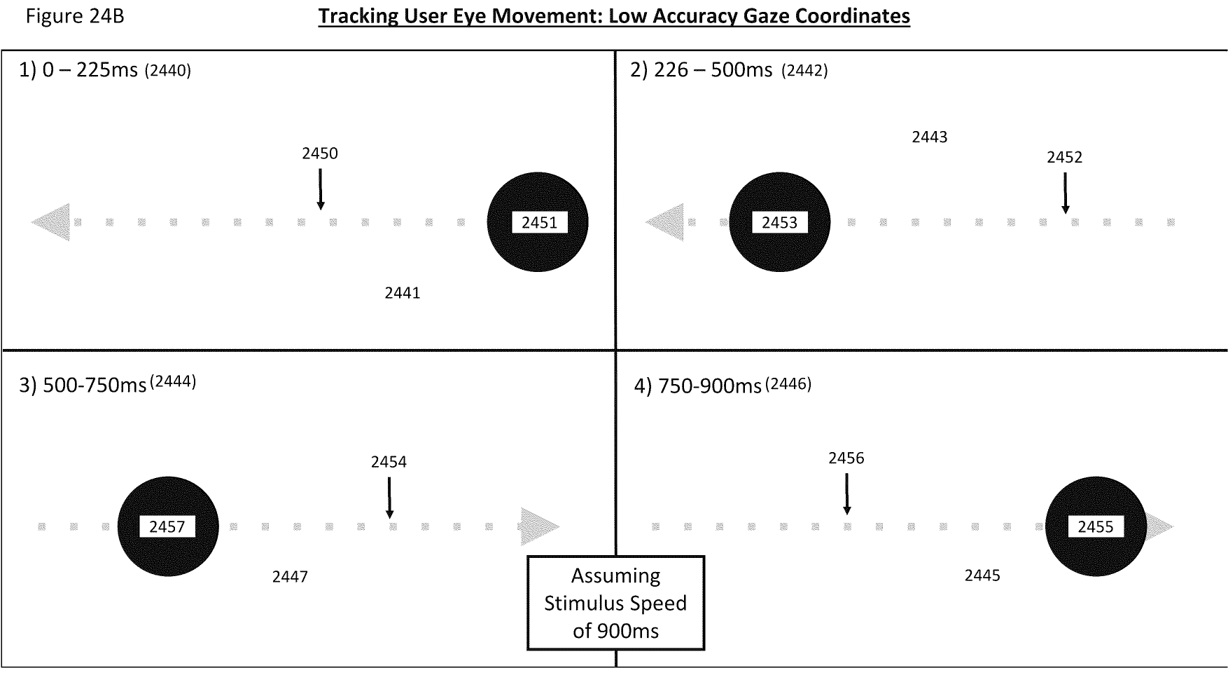

FIGS. 24A-24B show an exemplary eye tracking method in more detail. Eye tracking and/or other types of biometrics may be used to determine attentiveness of the user to the session and/or to the eye stimulus, such as a moving ball. To this end, measuring attentiveness in order to determine engagement during the eye-movements is preferably performed in order for the system to infer a reliable numerical score to determine the next appropriate step and when that step is to be executed, and in aggregate to calculate the degree of confidence with which the user has successfully completed a treatment session. Eye-Tracking analyzes a stream of webcam images and provides a determination, with degrees of confidence, as to the on-screen coordinates that correspond with the gaze of the user at a particular point in time.

The system as described herein may use these gaze coordinates in two ways to determine attentiveness. Though, there may be further ways to use digital ocular analysis in the app (I.E. pupil movements to diagnose PTSD). Two methods are shown in FIGS. 24A and 24B, one for highly accurate gaze tracking results and another for low accuracy results.

FIG. 24A shows an example of Tracking User Eye Movement: High Accuracy Gaze Coordinates. These measurements can be juxtaposed to the location of the eye stimulus or ball (non-limiting example of the previously described symbol, shown as reference numbers 2404, 2410, 2420 and 2428 in each of panels 1-4 respectively). The eye stimulus moves along the screen according to a tracking path (shown as reference numbers 2402, 2412, 2422 and 2424 in each of panels 1-4 respectively). The user's gaze preferably tracks or otherwise follows the eye stimulus as it moves along the tracking path.

When high confidence results are returned, the degree of proximity of the user's gaze to the location of the ball (eye stimulus) may be a primary indicator that the user is properly engaged. The gaze coordinates are represented as red dots on the figures below and indicated with reference numbers 2406, 2414, 2420 and 2426 in each of panels 1-4 respectively. Such gaze coordinates are preferably overlaid with the eye stimulus; at the very least, the x-axis coordinates of the gaze coordinates and the eye stimulus preferably align very closely. As an analogy, if the user's gaze was a laser, and the eye movement stimulus was a moving target, the user consistently hits the target throughout the treatment. The timings shown in each of panels 1-4 assume a stimulus speed of 900 ms.

When gaze coordinate scores are highly accurate, meaning the eye tracking system reports it has a high degree of confidence that it is a correct approximation, and the user is not able to hit the target (that is, their gaze is not properly focused on the target), preferably further analysis is performed to determine for example if there is some left to right eye motion happening, and/or if the user was totally distracted, either looking off screen, inconsistent eye motion or concentrated gaze on a localized area of the screen. The process for determining correct left to right eye motion that does not align with the stimulus is similar to the approach described in the low accuracy coordinates method with regard to FIG. 24B.

FIG. 24B shows an example of Tracking User Eye Movement: Low Accuracy Gaze Coordinates. There are many factors that can affect the confidence score of the gaze coordinates provided, including but not limited to the quality of the camera, how well lit the subject (user) is during treatment, whether the user is wearing glasses and so forth.

Low quality scores are not useless and may be assessed differently to get an indication of the user's attentiveness. In scenarios where the eye-tracking system cannot provide accurate coordinates, though the results are not necessarily accurate and cannot guarantee precisely what the user was definitively gazing at, they tend to exhibit somewhat predictable failure patterns when the user is following the stimulus with their eyes. When all the results are compared to each other, there is usually a clear left to right clustering of results, even if the coordinates are not reported to be in close proximity to the stimulus location at the time.

One exemplary non-limiting method for such an analysis is to take into account the speed setting the user has set, for the stimulus, which is the measure of time it takes for the stimulus to move from one side of the screen to the other side of the screen or display. When comparing the x-axis of each inaccurate grouping of results for each specified measure of time, half the results should have a statistically lower value for the second half-measure than it does the first. The method of analysis may then determine whether this pattern seems to hold for the duration of the eye-movement stimulus interaction.

For example, the eye stimulus is given reference numbers 2451, 2453, 2457 and 2455 in panels 1-4 respectively, and is shown moving along a travel path (reference numbers 2450, 2452, 2454 and 2456 in panels 1-4 respectively). However, the user's gaze cannot be determined accurately, and is shown as red dots 2441, 2443, 2447 and 2445 in panels 1-4 respectively. The above general localization method may be used instead.

Eye tracking (gaze tracking) as described herein, is preferably employed to determine attentiveness of the user and engagement with eye movements. Users are given instructions and suggestions for each set of EMs in a very strategic way as described with regard to FIGS. 23A-23E.

### Example—Process and Data

Without wishing to be limited to a single mode of operation, the software as described may be used according to the process described in this non-limiting Example, which provides a scripted approach that provides instruction to the user to encourage certain emotional responses before, during and after engaging in eye-motion stimulus, or eye movements (EM). Each stage of the treatment preferably features a variable set of at least 30 eye movements with specific accompanying emotional activity, referred to herein as “right brain”.

The treatment framework, as described in the scripting, in its current implementation features five distinct but seamlessly presented stages. The stages are designed to have specific right brain/emotional objectives, or intents, for the participant. The current embodiment of the treatment guides the participant through each stage by use of instructions/encouragements, self-provided feedback, auto-collected feedback, and sets of eye movement stimulus. The nature of everyone's trauma is all a bit different, as is each individual's response to the effects of that trauma. The guides are provided in a way that allows the participant to properly self-administer the treatment.

Each human is born with a fight/flight/freeze response. It is a network of neurons that comprise what is called the sympathetic nervous system. There are different theories about how PTSD is formed. One such theory, described herein without wishing to be limited by a single hypothesis, is that as something traumatic is occurring, sensory stimuli associated with it become connected to the original sympathetic neural network. For example, when someone is assaulted the sights, sounds, etc. that are experienced in those moments through sensory neurons form a new neural network that is in effect an extension of the original sympathetic nervous system network. It's a primitive way to protect oneself. The brain errs on the side of caution to promote survival, but quality of life can plummet when too many things are triggering. Because it is primitive, it is not precise. Seemingly random stimuli can set someone off when there is no real threat.

PTSD sufferers cannot turn off this aggravated response network voluntarily, despite the best efforts of generations of therapists who have tried to appeal to their patients' sense of logic. The left side of the brain is the province of memory, sequence (story), and cognition, however we submit that that entire side of the brain becomes disconnected from the trauma as an evolutionarily advantageous way for humans to instantly enter what has historically been an optimal state of action or reaction (and not thinking) in times of perceived threat. PTSD is a maladaptive mechanism in which a song on the radio can seem just as terrifying as a new dangerous circumstance. Almost all conventional therapies get their patients to tell their stories (that are incomplete) and put into words phenomena that are preverbal or even nonverbal. They want their patients to attain a more “integrated” experience that involves both sides of the brain. If the emotional networks and information can be paired with the logic, sequence, and context of the left brain, people will not have to be triggered by things that are actually innocuous. What has been missing is a way to thoroughly connect the two parts of the brain. Eye movement therapies have helped to fill this gap.

Whenever someone looks left, the right side of their brain activates. When they look right, sensory information crosses the corpus callosum, which is the only primary neural pathway between both sides to activate the left side of the brain. Normally the two hemispheres do not communicate with each other much at all. Francine Shapiro discovered that bilateral stimulation conduces whole brain synergistic function. She founded EMDR, which has patients move their eyes while recreating the worst events of their lives. There is not much structure to sessions other than free-association that will hopefully provide relief. Unfortunately such unstructured sessions require a skilled human therapist to administer; the extent of the therapeutic benefit depends on the skill of the therapist.

For the present invention, including with regard to the currently described implementation, these drawbacks to EMDR are overcome. Patients are given instructions and suggestions for each set of EMs in a very strategic way. The software, system and method as described herein helps users to recreate their trauma only one time (in order to access more deeply the neural network extension that is associated with it) as opposed to the unlimited amounts associated with other therapies, interface with their PTSD (their actual maladaptive automatic trauma response) in order to externalize and understand it, and imagine a different scenario that is not traumatic.

This last part of the method and system as described herein is believed to be strongly cathartic, again without wishing to be limited by a single hypothesis, because it links both hemispheres in this novel way, such that the aforementioned neural network extension that represents all of the sensory associations made during the traumatic event becomes deactivated and divorced from the original trauma network. Users do not lose their ability to protect themselves, nor memory of the trauma. They lose the unnecessary and debilitating effects of PTSD. This is only possible through the combination of traditional therapy goals with eye movements that are implemented carefully and strategically, which is supported by the present invention.

The software was tested in the form of a mobile telephone “app”. Of the first twenty-three (23) measured and monitored treatments:
86% (20 users) reported a positive symptom reduction
74% (17 users) reported a reliable symptom reduction (reduction of at least 5 points)
43% (10 users) reported a symptom change of 10 or greater

At least two patients initially reported an increased symptom change, and after consulting with a clinician, it was determined that the intent of the instruction was not understood. Following their second run of the treatment they recorded a dramatic decrease in their symptoms. This is a significant finding, because it demonstrates that simply repeatedly engaging in rapid eye-movements does not change the negative effects of PTSD for a user, while there is an apparent strong correlation of reduction in symptoms when the software instructions are understood, and the right brain is properly engaged in correlation to the REM in the treatment.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or stages manually, automatically, or a combination thereof. Moreover, according to actual instrumentation and equipment of preferred embodiments of the method and system of the present invention, several selected stages could be implemented by hardware or by software on any operating system of any firmware or a combination thereof. For example, as hardware, selected stages of the invention could be implemented as a chip or a circuit. As software, selected stages of the invention could be implemented as a plurality of software instructions being executed by a computer using any suitable operating system. In any case, selected stages of the method and system of the invention could be described as being performed by a data processor, such as a computing platform for executing a plurality of instructions.

Although the present invention is described with regard to a “computer” on a “computer network”, it should be noted that optionally any device featuring a data processor and/or the ability to execute one or more instructions may be described as a computer, including but not limited to a PC (personal computer), a server, a minicomputer. Any two or more of such devices in communication with each other, and/or any computer in communication with any other computer may optionally comprise a “computer network”.

1. A system for guiding a user during a treatment session for a mental health disorder, comprising a user computational device, the user computational device comprising a camera, a screen, a processor, a memory and a user interface, wherein said user interface is executed by said processor according to instructions stored in said memory, wherein eye movements of the user are tracked during the treatment session, and wherein the treatment session comprises a plurality of stages determined according to interactions of the user with said user interface and according to said tracked eye movements; wherein a timing, frequency and length of the treatment session is determined by the user through said user computational device, such that the user controls each treatment session; wherein said user computational device further comprises a display for displaying information to the user, and wherein said memory further stores instructions for performing eye tracking and instructions for providing an eye stimulus by being displayed on said display, and wherein said processor executes said instructions for providing said eye stimulus such that said eye stimulus is displayed on said display to the user, and for tracking an eye of said user; wherein said instructions further comprise instructions for adjusting said eye stimulus according to said eye tracking; wherein said instructions further comprise instructions for moving said eye stimulus from left to right, and from right to left, according to a predetermined speed and for a predetermined period; wherein said instructions further comprise instructions for determining said predetermined period according to one or more of a physiological reaction of the user, tracking said eye of the user and an input request of the user through said user interface.

2. (canceled)

3. (canceled)

4. (canceled)

5. The system of claim 4, wherein said predetermined period comprises a plurality of repetitions of movements of said eye stimulus from left to right, and from right to left.

6. The system of claim 5, wherein said instructions further comprise instructions for determining a degree of attentiveness of the user according to a biometric signal, and for adjusting moving said eye stimulus according to said degree of attentiveness.

7. The system of claim 6, wherein said biometric signal comprises heart rate, the system further comprising a heart rate monitor, wherein said heart rate monitor transmits heart rate information to said user computational device.

8. The system of claim 6 or 7, wherein said biometric signal comprises eye gaze, wherein said user computational device tracks eye gaze through said camera.

9. The system of claim 8, wherein said instructions further comprise instructions for determining whether said eye gaze tracking comprises high accuracy eye gaze tracking or low accuracy eye gaze tracking, and wherein said degree of attentiveness is calculated according to whether said eye gaze tracking comprises high accuracy eye gaze tracking or low accuracy eye gaze tracking, such that if said eye gaze tracking comprises high accuracy eye gaze tracking, said degree of attentiveness is determined according to a high degree of simultaneous overlap of locations of said eye gaze with locations of said eye stimulus; and alternatively wherein a lower extent of overlap of said locations is considered to calculate said degree of attentiveness.

10. The system of any of the above claims, wherein said instructions further comprise instructions for analyzing eye movements of the user according to one or more deep learning and/or machine learning algorithms.

11. The system of claim 10, wherein said instructions further comprise instructions for analyzing biometric information from the user according to one or more deep learning and/or machine learning algorithms.

12. The system of claim 11, wherein said one or more deep learning and/or machine learning algorithms comprise an algorithm selected from the group consisting of a DBN, a CNN and an RNN.

13. The system of claim 1, wherein the user computational device further comprises a user input device, wherein the user interacts with said user interface through said user input device to perform said interactions with said user interface during the treatment session.

14. The system of claim 1, further comprising a cloud computing platform, comprising a virtual machine, comprising a processor and a memory for storing a plurality of instructions; and a computer network, wherein said user computational device communicates with said cloud computing platform through said computer network; wherein said processor of said virtual machine executes said instructions on said memory to analyze at least biometric information of the user during a treatment session, and to return a results of said analysis to said user computational device for determining a course of said treatment session; wherein said biometric information is transmitted from a biometric measuring device directly to said cloud computing platform or alternatively is transmitted from said biometric measuring device to said user computational device, and from said user computational device to said cloud computing platform.

15. The system of claim 14, wherein said virtual machine analyses said biometric information from said biometric measuring device without input from said user computational device.

16. The system of claim 14, wherein said virtual machine analyses said biometric information from said biometric measuring device in combination with input from said user computational device.

17. The system of claim 16, wherein said biometric signal comprises heart rate, the system further comprising a heart rate monitor, wherein said cloud computing platform receives heart rate information directly or indirectly from said heart rate monitor.

18. The system of claim 17, wherein said biometric signal comprises eye gaze, wherein said user computational device obtains eye gaze information from said camera, and wherein said cloud computing platform receives said eye gaze information from said user computational device.

19. (canceled)

20. (canceled)

21. (canceled)

22. (canceled)

23. The system of claim 18, wherein said instructions of said virtual machine comprise instructions for determining a determining a degree of attentiveness of the user according to said tracking of eye movements.

24. The system of claim 1, wherein the mental health disorder comprises PTSD (post traumatic stress disorder), a phobia or a disorder featuring aspects of PTSD and/or a phobia.

25. A method of treatment of a mental health disorder, comprising operating the system of claim 1 by a user, and adjusting said plurality of stages in the treatment session to treat the mental health disorder.

26. The method of claim 25, comprising a plurality of treatment stages to be performed in the treatment session, said treatment stages comprising a plurality of eye movements from left to right and from right to left as performed by the user, according to a plurality of movements of an eye stimulus from left to right and from left to right; wherein an attentiveness of the user at each stage to said movements of said eye stimulus and wherein a subsequent stage is not started until sufficient attentiveness of the user to a current stage is shown.

27. The method of claim 26, wherein said treatment stages comprise at least Activation, wherein the user performs eye movements while considering a traumatic event; Externalization wherein the user performs eye movements while imagining themselves as a character outside of said traumatic event; and Deactivation, wherein the user performs eye movements while imagining such an event as non-traumatic.

28. The method of claim 27, wherein said treatment stages further comprise Reorientation, wherein the user performs eye movements while re-imagining the event.

29. The system of claim 1, further comprising a cloud computing platform for storing a plurality of scripts; and a computer network, wherein said user computational device communicates with said cloud computing platform through said computer network; wherein upon initiation of the treatment session, a script is accessed from said cloud computing platform by said user computational device; wherein said script is parsed into a plurality of frames, wherein each frame represents a graphical user interface (GUI) display for said user interface and wherein each frame is displayed through said display of said user computational device.

30. The system of claim 29, wherein one or more user commands for adjusting said script are provided through said user interface, and wherein said script is adjusted according to said one or more user commands.

31. The system of claim 1, further comprising a cloud computing platform, comprising a virtual machine, comprising a processor and a memory for storing a plurality of instructions; and a computer network, wherein said user computational device communicates with said cloud computing platform through said computer network; wherein said processor of said virtual machine executes said instructions on said memory for dynamic treatment generation configuration, for dynamically adjusting the treatment session according to an analysis of user interactions during the treatment session.

32. The system of claim 31, wherein said analysis of user interactions comprises receiving user feedback and adjusting the treatment session accordingly.

33. The system of claim 31, wherein said cloud computing platform further comprises a therapy session engine for receiving real time session data and for adjusting the treatment session accordingly.
